(19) United States
(12) Patent Application Publication
(10) Pub. No.: US 2025/0243272 A1
(43) Pub. Date: Jul. 31, 2025

(54) EPITOPE OF REGULATORY T CELL SURFACE ANTIGEN, AND ANTIBODY SPECIFICALLY BINDING THERETO

(71) Applicant: GOOD T CELLS, INC., Seoul (KR)

(72) Inventors: Jung Ho Kim, Seoul (KR); Beom Seok Kim, Seoul (KR)

(21) Appl. No.: 17/758,550

(22) PCT Filed: Nov. 9, 2020
(86) PCT No.: PCT/KR2020/015656

§ 371 Date: Jul. 8, 2022

(30) Foreign Application Priority Data
Nov. 8, 2019 (KR) 10-2019-0142251

(51) Int. Cl. C07K 16/28; A61K 39/00; A61K 47/68; A61P 35/00; C12N 15/85; G01N 33/68

(52) U.S. Cl. CPC C07K 16/2803; A61K 47/6849; A61P 35/00; C12N 15/85; G01N 33/6857; A61K 2039/505; C07K 2317/34; C07K 2317/565; G01N 2333/70503

(57) ABSTRACT

The present invention relates to an epitope of Lrig-1 (leucine-rich repeats and immunoglobulin-like domains 1) protein, which is an antigen present on the surface of a regulatory T cell, and an antibody or antigen-binding fragment that binds specifically thereto.

### CROSS-REFERENCE TO RELATED APPLICATIONS

This application is a U.S. National Stage application of International Patent Application no. PCT/KR2020/015656, filed Nov. 9, 2020, which claims the benefit of Korean Patent Application no. 10-2019-0142251, filed Nov. 8, 2019.

### SEQUENCE LISTING

A computer readable form of the Sequence Listing is submitted via a USPTO patent electronic filing system with this application and is incorporated into this application by reference in its entirety. The Sequence Listing is contained in the sequence listing text file created on Apr. 26, 2023, having the file name “22-1137-WO-US_SequenceListing_ST25.txt” and being 139 kb in size.

### TECHNICAL FIELD

The present invention relates to an epitope of Lrig-1 (leucine-rich and immunoglobulin-like domains 1) protein, which is an antigen present on the surfaces of regulatory T cells, and an antibody or antigen-binding fragment that binds specifically thereto.

### BACKGROUND ART

One of the most remarkable properties of every normal individual's immune system is its ability to recognize and eliminate non-self antigens while not reacting harmfully to that of self-antigenic substances. Such unresponsiveness to self-antigens is called immunologic unresponsiveness or tolerance. Self-tolerance occurs either by elimination of lymphocytes that may have specific receptors for self-antigens or by inactivation of self-reactive lymphocytes after contact with self-antigens. Abnormalities in the induction or maintenance of self-tolerance lead to immune responses against self-antigens, which may result in disorders called autoimmune diseases.

For the treatment of the autoimmune disease, the concept of suppressor T cells with the possibility of presence of T cells capable of controlling and suppressing the effector function of conventional T cells was first proposed by Gershon in the early 1970s (R. K. Gershon and K. Kondo, Immunology, 1970, 18:723-37). Since then, research has been conducted to elucidate the biological properties and functions of regulatory T cells in many fields of immunology

Accordingly, the regulatory T cells (Treg) play an important role in naturally preventing the occurrence of excessive inflammation and immune responses, but it has been reported that, when autoimmune diseases and chronic inflammatory diseases occur, the function and number of regulatory T cells remarkably decrease. Thus, for patients with immune diseases and inflammatory diseases, it is important that regulatory T cells are generated at normal levels, which can be one of methods for treatment of these diseases.

Until now, studies on genes and proteins present specifically in regulatory T cells have been conducted, suggesting that substances such as CD25, CTLA4, CD62L, CD38, CD103, GITR, and CD45RB may correspond to marker substances. However, genes or proteins that can target regulatory T cells alone have not yet been reported.

Meanwhile, the variable domain of an antibody includes three hypervariable regions, called complementarity determining regions (hereinafter referred to as “CDRs”), and four framework regions. The CDRs mainly serve to bind to an antigenic determinant (epitope) of an antigen. The CDRs of each chain are typically referred to as CDR1, CDR2, and CDR3, numbered sequentially starting from the N-terminus, and are also typically identified by the chain in which the particular CDR is located.

### DISCLOSURE

### Technical Problem

An object of the present invention is to provide an epitope of Lrig-1 (leucine-rich and immunoglobulin-like domains 1) protein present on the surface of a regulatory T cells (Treg cell).

Another object of the present invention is to provide an antibody or antigen-binding fragment capable of binding specifically to the epitope.

Still another object of the present invention is to provide a pharmaceutical composition for preventing or treating cancer containing, as an active ingredient, the antibody or antigen-binding fragment capable of specifically binding to the epitope.

Yet another object of the present invention is to provide an antibody-drug conjugate (ADC) in which a drug, for example, an anticancer drug, is conjugated to the antibody.

Still yet another object of the present invention is to provide a pharmaceutical composition for preventing or treating cancer containing the antibody-drug conjugate as an active ingredient.

A further object of the present invention is to provide a method of preventing or treating cancer using the antibody or antigen-binding fragment capable of specifically binding to the epitope, and the antibody-drug conjugate.

However, objects to be achieved by the present invention are not limited to the objects mentioned above, and other objects not mentioned herein will be clearly understood by those of ordinary skill in the art from the following description.

### Technical Solution

The present invention is directed to an epitope of Lrig-1 (leucine-rich and immunoglobulin-like domains 1) protein or an antibody or antigen-binding fragment that binds specifically to the epitope.

In the present invention, the “Lrig-1 protein” is a transmembrane protein present on the surfaces of regulatory T cells, and is composed of leucine-rich repeats (LRRs) and three immunoglobulin-like domains on the extracellular or lumen side, a cell transmembrane sequence, and a cytoplasmic tail region. The LRIG gene family includes LRIG1, LRIG2, and LRIG3, and the amino acids constituting each member of the family are highly conserved. The LRIG1 gene is highly expressed in normal skin, and can be expressed in basal and hair follicle cells to regulate proliferation of epithelial stem cells. Thus, the LRIG1 gene plays an important role in maintaining homeostasis of the epidermis, and its absence may lead to psoriasis or skin cancer. It has been reported that a deletion of chromosome region 3p14.3 in which LRIG1 is located is likely to develop into cancer cells. In fact, it was found that expression of LRIG1 greatly decreased in renal cell carcinoma and cutaneous squamous cell carcinoma. In recent years, it has also been found that Lrig-1 is expressed in only about 20 to 30% of cancers. Meanwhile, for the purpose of the present invention, the Lrig-1 protein may be a protein present in humans or mice, without being limited thereto.

In one embodiment of the present invention, the Lrig-1 protein may be a Lrig-1 protein derived from mammals, including primates such as humans and monkeys, rodents such as mice and rats, and the like.

In one embodiment of the present invention, the Lrig-1 protein may be a human-derived Lrig-1 protein represented by SEQ ID NO: 1, which may be encoded by the nucleic acid sequence represented by SEQ ID NO: 2, without being limited thereto.

In another embodiment of the present invention, the Lrig-1 protein may be a mouse-derived Lrig-1 protein represented by SEQ ID NO: 3, which may be encoded by the nucleic acid sequence represented by SEQ ID NO: 4, without being limited thereto.

In another embodiment of the present invention, the Lrig-1 protein may be an extracellular domain of the Lrig-1 protein, without being limited thereto.

The Lrig-1 extracellular domain in the present invention may be an extracellular domain of Lrig-1 protein derived from mammals including primates such as humans and monkeys, rodents such as mice and rats, and the like. For the purpose of the present invention, the extracellular protein of the Lrig-1 protein may be an extracellular domain of Lrig-1 protein derived from a human or mouse, without being limited thereto.

In one embodiment of the present invention, the extracellular domain of the Lrig-1 protein may be represented by SEQ ID NO: 5, which corresponds to amino acid positions 35 to 794 in the amino acid sequence of the human-derived Lrig-1 protein, without being limited thereto.

In another example of the present invention, the extracellular domain of the Lrig-1 protein may be represented by SEQ ID NO: 6, which corresponds to amino acid positions 35 to 794 in the amino acid sequence of the mouse-derived Lrig-1 protein, without being limited thereto.

Hereinafter, the present invention will be described in more detail.

### 1. Epitope of Lrig-1 Protein

According to one embodiment of the present invention, the epitope of the Lrig-1 protein may consist of some amino acids (e.g., 2 to 50, 6 to 45, or 10 to 44 amino acids) of the Lrig-1 protein represented by SEQ ID NOs: 1, 3, or 5, without being limited thereto. Another embodiment of the present invention provides an epitope of Lrig-1 protein comprising at least one polypeptide selected from the group consisting of polypeptides consisting of the amino acid sequence represented by SEQ ID NOs: 7 to 70.

In one embodiment of the present invention, the epitope of the Lirg-1 protein may be an epitope comprising a polypeptide consisting of the amino acid sequence represented by SEQ ID NO: 71 or SEQ ID NO: 72, without being limited thereto.

The epitope of the present invention may be a conformational epitope.

The “conformational epitope” of the present invention consists of a discontinuous amino acid sequence, unlike a one-dimensional linear epitope consisting of a continuous sequence. This conformational epitope reacts with the three-dimensional structure of the antigen-binding site of an antibody.

### 2. Nucleic Acid Molecule Encoding an Epitope of Lrig-1 Protein

Another embodiment of the present invention provides a nucleic acid molecule encoding the epitope provided in the present invention.

Nucleic acid molecules of the present invention include all nucleic acid molecules having polynucleotide sequences which are translated into the amino acid sequences of the polypeptides provided by the present invention, as known to those skilled in the art. Therefore, various polynucleotide sequences with open reading frames (ORFs) may be produced, which are also all included in the nucleic acid molecules of the present invention.

3. Expression Vector into which Nucleic Acid Molecule Encoding Epitope of Lrig-1 Protein has been Inserted

Another embodiment of the present invention provides an expression vector into which the isolated nucleic acid molecule provided in the present invention has been inserted.

In the present invention, the “vector” is any nucleic acid molecule capable of transporting another nucleic acid linked thereto. One type of vector is a “plasmid,” which refers to a circular double-stranded DNA to which an additional DNA segment may be ligated. Another type of vector is a phage vector. Yet another type of vector is a viral vector, where an additional DNA segment can be ligated into the viral genome. Certain vectors are capable of autonomous replication in a host cell into which they have been introduced (for example, bacterial vectors having a bacterial origin of replication are episomal mammalian vectors). Other vectors (e.g., non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, the terms “plasmid” and “vector” may be used interchangeably as the plasmid is the most commonly used form of vector.

Specific examples of the expression vector in the present invention may be selected from the group consisting of commercially widely used pCDNA vectors, F, R1, RP1, Col, pBR322, ToL, Ti vectors; cosmids; phages such as lambda, lambdoid, M13, Mu, p1 P22, Qμμ, T-even, T2, T3, T7; and plant viruses, without being limited thereto. Any expression vector known as expression vectors to those skilled in the art may be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation, without being limited thereto, and those skilled in the art may adopt and use an introduction method appropriate for the expression vector and the host cell which are used. The vector may preferably contain at least one selection marker, without being limited thereto, and selection can be performed using a vector that contains no selection marker, depending on whether or not a product is produced. The selection marker is chosen depending on the target host cell, which is done using methods already known to those skilled in the art, and thus the present invention has no limitation thereon.

In order to facilitate purification of the protein encoded by the nucleic acid molecule of the present invention, a tag sequence may be inserted into and fused to the expression vector. Examples of the tag include, but are not limited to, hexa-histidine tag, hemagglutinin tag, myc tag, or flag tag, and any tag known to those skilled in the art which facilitates purification may be used in the present invention.

4. Host Cell Line Transformed with Expression Vector into which Nucleic Acid Molecule Encoding Epitope of Lrig-1 Protein has been Inserted

Another embodiment of the present invention provides a host cell line transformed with the expression vector provided in the present invention.

In the present invention, the term “host cell” includes individual cells or cell cultures which may be or have been recipients of the vector(s) for incorporation of a polypeptide insert. The host cells include progeny of a single host cell, and the progeny may not necessarily be completely identical (in morphology or in genomic DNA complement) to the original parent cell due to natural, accidental, or intentional mutation. The host cells include cells transfected in vivo with the polynucleotide(s) of the present invention.

In the present invention, the host cells may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells, without being limited thereto, any cell type known to those skilled in the art which may be used as a host cell line may be used.

The transformation method of the present invention is any method of introducing a desired vector into the host cell, and may include any known method capable of introducing the vector into the host cell. For example, a method using CaCl₂), electroporation, microinjection, calcium phosphate precipitation, electroporation, liposome-mediated transfection, DEAE-dextran treatment, gene bombardment, and transfection using virus, may be used, without being limited thereto.

### 5. Antibody or Antigen-Binding Fragment

Another embodiment of the present invention provides an antibody or antigen-binding fragment that binds specifically to an epitope of the present invention.

In the present invention, the antibody is a full-length antibody or a part of the antibody, has the ability to bind to the Lrig-1 protein, and includes any antibody fragment that binds to the Lrig-1 antigen determining region in a manner competitive with the binding molecule of the present invention.

In the present invention, the term “antibody” refers to a protein molecule which serves as a receptor that specifically recognizes an antigen, including an immunoglobulin molecule that is immunologically reactive with a particular antigen. For the purpose of the present invention, the antigen may be Lrig-1 protein present on the surfaces of regulatory T cells. Preferably, the antibody may specifically recognize the leucine-rich region or immunoglobulin-like domain of the Lrig-1 protein, without being limited thereto.

In the present invention, the “immunoglobulin” has a heavy chain and a light chain, and each of the heavy chain and the light chain comprises a constant region and a variable region. The variable region of each of the light chain and the heavy chain contains three hypervariable regions, called complementarity determining regions (hereinafter referred to as “CDRs”), and four framework regions. The CDRs primarily serve to bind to an antigenic determinant (epitope) of an antigen. The CDRs of each chain are typically referred to as CDR1, CDR2, and CDR3, numbered sequentially starting from the N-terminus, and are also typically identified by the chain in which the particular CDR is located.

In the present invention, the “full-length antibody” has a structure having two full-length light chains and two full-length heavy chains, wherein each of the light chains is linked to the heavy chain via a disulfide bond. Examples of the full-length antibody include IgA, IgD, IgE, IgM, and IgG. Subtypes of the IgG include IgG1, IgG2, IgG3, and IgG4.

In addition, as used herein, the term “antigen-binding fragment” refers to a fragment having an antigen-binding function. Examples of the antigen-binding fragment include: (1) a Fab fragment consisting of a light chain variable region (VL), a heavy chain variable region (VH), a light chain constant region (CL), and a heavy chain constant region 1 (CH1); (2) a Fd fragment consisting of VH and CH1 domains; (3) a Fv fragment consisting of VL and VH domains of a single antibody; (4) a dAb fragment consisting of a VH domain; (5) an isolated CDR region; (6) a F(ab′)₂ fragment which is a bivalent fragment including two linked Fab fragments; (7) a single-chain Fv molecule (scFv) in which a VH domain and a VL domain are linked together by a peptide linker that allows the two domains to associate to form an antigen-binding site; (8) a bispecific single-chain Fv dimer; and (9) a diabody which is a multivalent or multispecific fragment constructed by gene fusion. The antigen-binding fragment may be obtained as a Fab or F(ab′)₂ fragment when a proteolytic enzyme, for example, papain or pepsin is used, and the antigen-binding fragment may be produced through a genetic recombination technique.

In addition, in the present invention, the antibody may be, without limitation, a monoclonal antibody, a polyclonal antibody, a chimeric antibody, a humanized antibody, a human antibody, a bivalent, bispecific molecule, a minibody, a domain antibody, a bispecific antibody, an antibody mimetic, a unibody, a diabody, a triabody, a tetrabody, or a fragment thereof.

In addition, as used herein, the term “monoclonal antibody” refers to an antibody molecule having a single molecular composition, which is obtained from a population of substantially identical antibodies, and exhibits single binding specificity and affinity for a particular epitope.

In the present invention, the “chimeric antibody” is an antibody which is obtained by recombination of a variable region of a mouse antibody and a constant region of a human antibody, and has a greatly improved immune response compared to the mouse antibody.

In addition, as used herein, the term “humanized antibody” refers to an antibody obtained by modifying the protein sequence of an antibody of non-human species origin so that the protein sequence is similar to an antibody variant naturally produced in humans. For example, the humanized antibody may be produced by recombining mouse-derived CDRs with a human antibody-derived FR to obtain a humanized variable region, and recombining the humanized variable region with a constant region of a desired human antibody.

As used herein, the term “binding” or “specific binding” refers to the affinity of the antibody or antibody composition of the present invention for an antigen. In antigen-antibody binding, the “specific binding” is distinguishable from non-specific background binding, typically when the dissociation constant (Kd) is less than 1×10⁻⁵ M, less than 1×10⁻⁶ M, or less than 1×10⁻⁷M. Specific binding can be detected by methods known in the art, such as ELISA, surface plasmon resonance (SPR), immunoprecipitation, and coprecipitation, which include an appropriate control that can distinguish between non-specific binding and specific binding.

The antibody or antigen-binding fragment of the present invention may exist as a multimer, such as a dimer, a trimer, a tetramer, or a pentamer, which includes at least a portion of the antigen-binding activity of a monomer. Such multimers also include a homomultimer or a heteromultimer. Antibody multimers contain a large number of antigen-binding sites, and thus have excellent antigen-binding activity compared to monomers. Antibody multimers are also easily used to produce multifunctional (bifunctional, trifunctional or tetrafunctional) antibodies.

As used herein, the term “multifunctional” refers to an antibody or antigen-binding fragment which has two or more activities or functions (e.g., antigen-binding activity, enzymatic activity, and ligand- or receptor-binding activity). For example, the antibody of the present invention may bind to a polypeptide having enzymatic activity, such as luciferase, acetyltransferase, or galactosidase. Multifunctional antibodies also include multivalent or multispecific (e.g., bispecific, trispecific, etc.) forms of antibodies.

### 6. Pharmaceutical Composition Containing Antibody or Antigen-Binding Fragment as Active Ingredient

In addition, the antibody according to the present invention may bind specifically to an epitope comprising a polypeptide represented by any one of SEQ ID NOs: 7 to 72 present on regulatory T cells, thereby activating, maintaining or inhibiting the function of the regulatory T cells to regulate the activity of effector T cells, thereby preventing or treating cancer.

In the present invention, the term “cancer” indicates or refers to a physiological condition typically characterized by unregulated cell growth in mammals. Cancer to be prevented, ameliorated or treated in the present invention may be a solid tumor consisting of an agglomerate generated by abnormal cell growth in a solid organ, and may be, without limitation, gastric cancer, liver cancer, glioblastoma, ovarian cancer, colorectal cancer, prostate cancer, pancreatic cancer, melanoma, or lung cancer, depending on the location of the solid organ.

### 7. Antibody-Drug Conjugate (ADC)

Another embodiment of the present invention provides an antibody-drug conjugate (ADC) comprising: the antibody or antigen-binding fragment provided by the present invention; and a drug.

As used herein, the term “antibody-drug conjugate (ADC)” refers to a form in which the drug and the antibody are chemically linked to each other without degrading the biological activities of the antibody and the drug. In the present invention, the antibody-drug conjugate refers to a form in which the drug is conjugated to the N-terminal amino acid residue of the heavy and/or light chain of the antibody, specifically, a form in which the drug is conjugated to the N-terminal α-amine group of the heavy and/or light chain of the antibody.

As used herein, the “drug” may mean any substance having a certain biological activity for a cell, which is a concept including DNA, RNA, or a peptide. The drug may be in a form containing a reactive group capable of reacting and crosslinking with an α-amine group, and also includes a form to which a linker containing a reactive group capable of reacting and crosslinking with an α-amine group is connected.

In the present invention, examples of the reactive group capable of reacting and crosslinking with the α-amine group include, without particular limitation, any amine-reactive groups known in the art, which are capable of reacting and crosslinking with the N-terminal α-amine group of the heavy or light chain of the antibody. The reactive group may, for example, be any one of isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester, without being limited thereto.

In the present invention, the antibody-drug conjugate comprises, as the antibody or antigen-binding fragment, an antibody or antigen-binding fragment that binds specifically to the epitope of the present invention, that is, an epitope of Lrig-1 protein, or an epitope comprising a polypeptide consisting of some amino acids of the amino acid sequence of the extracellular domain of the Lrig-1 protein, or an epitope comprising a polypeptide consisting of the amino acid sequence represented by Formula 1, or an epitope comprising a polypeptide represented by the amino acid sequence of any one of SEQ ID NOs: 48 to 113, wherein the drug may be any drug capable of treating a disease targeted by the Lrig-1 antibody, for example, an anticancer drug capable of treating cancer, without being limited thereto.

In one embodiment of the present invention, the anticancer drug may be, without limitation, any drug that may be used for the prevention, amelioration or treatment of cancer. For example, the anticancer drug may be selected from the group consisting of nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, capecitabine, decitabine, mercaptopurine, thioguanine, cladribine, carmofur, raltitrexed, docetaxel, paclitaxel, irinotecan, belotecan, topotecan, vinorelbine, etoposide, vinblastine, idarubicin, mitomycin, bleomycin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminoglutethimide, anagrelide, olaparib, navelbine, fadrozole, tamoxifen, toremifene, testolactone, anastrozole, letrozole, vorozole, bicalutamide, lomustine, 5FU, vorinostat, entinostat, and carmustine, without being limited thereto.

### 8. Pharmaceutical Composition Containing Antibody-Drug Conjugate (ADC) as Active Ingredient

Another embodiment of the present invention provides a pharmaceutical composition for preventing or treating cancer containing, as an active ingredient, the antibody or antigen-binding fragment provided by the present invention, or the antibody-drug conjugate (ADC).

In the present invention, the antibody or antigen-binding fragment or the antibody-drug conjugate obtained by conjugating the drug thereto, which is contained in an active ingredient in the pharmaceutical composition, may bind specifically to an epitope comprising a polypeptide represented by any one of the amino acid sequences of SEQ ID NOs: 7 to 72, thereby inhibiting the function of regulatory T cells and maintaining or increasing the activity of effector T cells, thereby very effectively treating cancer.

The cancer in the present invention may be a solid tumor consisting of an agglomerate generated by abnormal cell growth in a solid organ. In a specific embodiment, the cancer may be, without limitation, gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, or lung cancer, depending on the location of the solid organ.

### 9. Others

Meanwhile, in the present invention, “prevention” may include, without limitation, any action that blocks the symptoms of a disease or suppresses or delays the symptoms of the disease by using the pharmaceutical composition of the present invention.

In addition, in the present invention, “treatment” may include, without limitation, any action that alleviates or beneficially alters symptoms of a disease by using the pharmaceutical composition of the present invention.

In the present invention, the pharmaceutical composition may be in the form of capsules, tablets, granules, injections, ointments, powders, or beverages, and the pharmaceutical composition may be for administration to humans.

In the present invention, for use, the pharmaceutical composition may be formulated in the form of oral preparations such as powders, granules, capsules, tablets, and aqueous suspensions, preparations for external use, suppositories, and sterile injectable solutions, according to the respective conventional methods, without being limited thereto. The pharmaceutical composition of the present invention may further contain pharmaceutically acceptable carriers. As the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preserving agent, a pain-relieving agent, a solubilizer, an isotonic agent, a stabilizer, and the like may be used for injection; and a base, an excipient, a lubricant, a preserving agent, and the like may be used for topical administration. In addition, the pharmaceutical composition of the present invention may be prepared in various dosage forms by being mixed with the pharmaceutically acceptable carriers as described above. For example, for oral administration, the pharmaceutical composition may be formulated in the form of tablets, troches, capsules, elixirs, suspensions, syrups, wafers, or the like. For injection, the pharmaceutical composition may be formulated in the form of unit dosage ampoules or in multiple-dosage forms. In addition, the pharmaceutical composition may be formulated into solutions, suspensions, tablets, capsules, sustained-release preparations, or the like.

Meanwhile, examples of carriers, diluents or excipients suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. In addition, a filler, an anti-coagulant, a lubricant, a wetting agent, a fragrance, an emulsifier, a preservative, and the like may further be contained.

The routes of administration of the pharmaceutical composition of the present invention include oral, intravenous, intramuscular, intraarterial, intramedullary, intradural, intracardiac, transdermal, subcutaneous, intraperitoneal, intranasal, intestinal, topical, sublingual, or rectal routes, without being limited thereto. Oral or parenteral administration is preferred.

In the present invention, the “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present invention may also be formulated as suppositories for intrarectal administration.

The pharmaceutical composition of the present invention may vary depending on a variety of factors, including the activity of a specific compound used, the patient's age, body weight, general health status, sex and diet, the time of administration, the route of administration, the rate of excretion, drug combination, and the severity of a specific disease to be prevented or treated. The dosage of the pharmaceutical composition may vary depending on the patient's condition and body weight, the severity of disease, the drug form, and the route and duration of administration, but may be appropriately selected by those skilled in the art, and the pharmaceutical composition may be administered at a dose of 0.0001 to 50 mg/kg/day or 0.001 to 50 mg/kg/day. This administration may be done once a day or several times a day. The dose is not intended to limit the scope of the present invention in any way. The pharmaceutical composition according to the present invention may be formulated in the form of pills, sugar-coated tablets, capsules, liquids, gels, syrups, slurries, or suspensions.

### 10. Method for Treatment

Another embodiment of the present invention is directed to a method for a method for preventing or treating cancer comprising a step of administering, to a subject, the antibody or antigen-binding fragment or the antibody-drug conjugate (ADC) according to the present invention.

In the present invention, the term “cancer” indicates or refers to a physiological condition typically characterized by unregulated cell growth in mammals. Cancer to be prevented, ameliorated or treated in the present invention may be a solid tumor consisting of an agglomerate generated by abnormal cell growth in a solid organ, and may be, without limitation, gastric cancer, liver cancer, glioblastoma, ovarian cancer, colorectal cancer, prostate cancer, pancreatic cancer, melanoma, or lung cancer, depending on the location of the solid organ.

The antibody or antigen-binding fragment or antibody-drug conjugate of the present invention may bind specifically to an epitope comprising a polypeptide represented by the amino acid sequence of any one of SEQ ID NOs: 7 to 72, thereby inhibiting the function of regulatory T cells and maintaining or increasing the activity of effector T cells, thereby very effectively treating cancer.

As used herein, the term “subject” refers to a subject suspected of developing cancer, and the subject suspected of developing cancer means mammals, including humans, mice, and domestic animals, that have developed or is likely to develop the disease in question. However, the term includes, without limitation, any subject that may be treated with the antibody or antibody-drug conjugate of the present invention.

The method of the present invention may comprise administering a pharmaceutically effective amount of the antibody or the antibody-drug conjugate. The suitable total daily dose of the antibody or the antibody-drug conjugate may be determined by an attending physician or veterinarian within the scope of sound medical judgment, and the antibody or the antibody-drug conjugate may be administered once or several times. However, for the purposes of the present invention, a specific therapeutically effective amount for a particular patient is preferably applied differently depending on various factors, including the type and degree of reaction to be achieved, the specific composition, including whether other agents are used as the case may be, the patient's age, body weight, general health status, sex, and diet, the time of administration, the route of administration, the secretion rate of the composition, the duration of treatment, and drugs used simultaneously or in combination with the specific composition, and similar factors well known in the medical field.

Meanwhile, the method for preventing or treating cancer may be, without limitation, a combination therapy that further comprises administering a compound or substance having therapeutic activity against one or more cancer diseases.

In the present invention, the “combination” should be understood to refer to simultaneous, individual, or sequential administration. When the administration is done in a sequential or individual manner, the second component should be administered at intervals such that beneficial effects of the combination are not lost.

In the present invention, the dosage of the antibody or antibody-drug conjugate may be about 0.0001 μg to 500 mg per kg of patient's body weight, without being limited thereto.

### Advantageous Effects

The antibody or antigen-binding fragment that binds specifically to the epitope of Lrig-1 protein according to the present invention may bind specifically to the epitope of the present invention, which is present on regulatory T cells, thereby inhibiting the function of the regulatory T cells and maintaining or increasing the activity of effector T cells. Thus, it may be very efficiently used for the prevention, amelioration or treatment of cancer.

### BRIEF DESCRIPTION OF DRAWINGS

FIG. 1 shows the structure of Lrig-1 protein according to one embodiment of the present invention.

FIG. 2 shows the structure of Lrig-1 protein according to one embodiment of the present invention.

FIG. 3 shows the expression level of Lrig-1 mRNA measured according to one example of the present invention.

FIG. 4 shows the expression level of Lrig-1 mRNA measured according to one example of the present invention.

FIG. 5 shows the expression level of Lrig-1 mRNA measured according to one example of the present invention.

FIG. 6 shows the expression levels of Lrig-1, Lrig-2, and Lrig-3 mRNAs measured according to one example of the present invention.

FIG. 7 shows the results of comparing the expression levels of Lrig-1 protein in regulatory T cells and non-regulatory T cells, measured according to one example of the present invention.

FIG. 8 shows expression of Lrig-1 protein on the surfaces of regulatory T cells, measured according to one example of the present invention.

FIG. 9 shows the results of analyzing the binding affinities of antibodies (GTC210-01, GTC210-02, GTC210-03, GTC210-04, GTC110-01, GTC110-02, GTC110-03 and GTC110-04) for Lrig-1 protein in one example of the present invention.

FIG. 10 shows the results of analyzing the mechanisms of regulating Lrig-1 protein-induced Stat3 phosphorylation in regulatory T cells by Lrig-1 protein-specific monoclonal antibodies (GTC210-01, GTC210-02, GTC210-03, GTC210-04, GTC110-01, GTC110-02, GTC110-03 and GTC110-04) in one example of the present invention.

FIG. 11 shows the experimental design of cancer treatment using Lrig-1 protein-specific monoclonal antibodies in one example of the present invention.

FIG. 12 shows cancer therapeutic effects obtained using Lrig-1 protein-specific monoclonal antibodies (GTC110-01, GTC110-02, GTC110-03 and GTC110-04) in one example of the present invention.

FIGS. 13 to 15 show conditions for mass spectrometry according to one example of the present invention.

FIGS. 16 and 17 show the results of covalent labeling mass spectrometry (MS) performed using chymotrypsin for the extracellular domain of Lirg-1 protein in one example of the present invention.

FIGS. 18 and 19 show the results of covalent labeling mass spectrometry (MS) performed using trypsin for the extracellular domain of Lirg-1 protein in one example of the present invention.

FIGS. 20 and 21 show the results of covalent labeling mass spectrometry (MS) performed using Asp-N+Lys-C for the extracellular domain of Lirg-1 protein in one example of the present invention.

FIG. 22 shows the results of crosslinking mass spectrometry (MS) performed using chymotrypsin for the extracellular domain of Lirg-1 protein in one example of the present invention.

FIG. 23 shows the results of crosslinking mass spectrometry (MS) performed using trypsin for the extracellular domain of Lirg-1 protein in one example of the present invention.

FIG. 24 shows the results of crosslinking mass spectrometry (MS) performed using Asp-N+Lys-C for the extracellular domain of Lirg-1 protein in one example of the present invention.

FIG. 25 shows an epitope region of the extracellular domain of Lrig-1 protein, to which a GTC110-04 antibody specifically binds, in an example of the present invention.

### BEST MODE

The present invention is directed to an epitope of Lrig-1 (leucine-rich and immunoglobulin-like domains 1) protein or an antibody or antigen-binding fragment that specifically binds to the epitope.

In one embodiment of the present invention, the extracellular domain of the Lrig-1 protein may be represented by SEQ ID NO: 5, which corresponds to amino acid positions 35 to 794 in the amino acid sequence of the human-derived Lrig-1 protein, without being limited thereto.

In another example of the present invention, the extracellular domain of the Lrig-1 protein may be represented by SEQ ID NO: 6, which corresponds to amino acid positions 35 to 794 in the amino acid sequence of the mouse-derived Lrig-1 protein, without being limited thereto.

### MODE FOR INVENTION

Hereinafter, the present invention will be described in more detail with reference to examples. These examples are only for illustrating the present invention in more detail, and it will be obvious to those of ordinary skill in the art that the scope of the present invention according to the subject matter of the present invention is not limited by these examples.

### EXAMPLES

### [Preparation Example 1] T Cell Subset Culture

In order to examine whether the Lrig-1 protein is expressed only in regulatory T cells (Treg) cells, Th0, Th1, Th2, Th17 and iTreg, which are T cell subsets, were prepared. The iTreg refers to cells whose differentiation has been artificially induced in a medium having the following composition, unlike nTreg which has been naturally isolated.

The T cell subsets were obtained by isolating naïve T cells from mouse spleens, and then inducing the isolated cells to differentiate into each T cell subset by 72 hours of culture in RPMI1640 (Invitrogen Gibco, Grand Island, NY) nutrient medium, which contained 10% fetal bovine serum (FBS; hyclone, logan, UT) and further contained the components shown in Table 1 below, in an incubator at 37° C. under 5% CO₂.

TABLE 1

| Differentiated cells | Composition |
|---|---|
| Th0 | anti-CD3 and anti-CD28 |
| Th1 | IL-12 and anti-IL-4 antibody |
| Th2 | IL-4 and anti-IFNβ |
| Th17 | IL-6, TGFβ, anti-IFNβ and anti-IL-4 |
| iTreg | IL-2 and TGFβ |

### [Example 1] Structural Analysis of Lrig-1

The three-dimensional steric structure of the extracellular domain of Lrig-1 protein was predicted to produce an antibody specific for the Lrig-1 protein, a protein present on the surfaces of regulatory T cells.

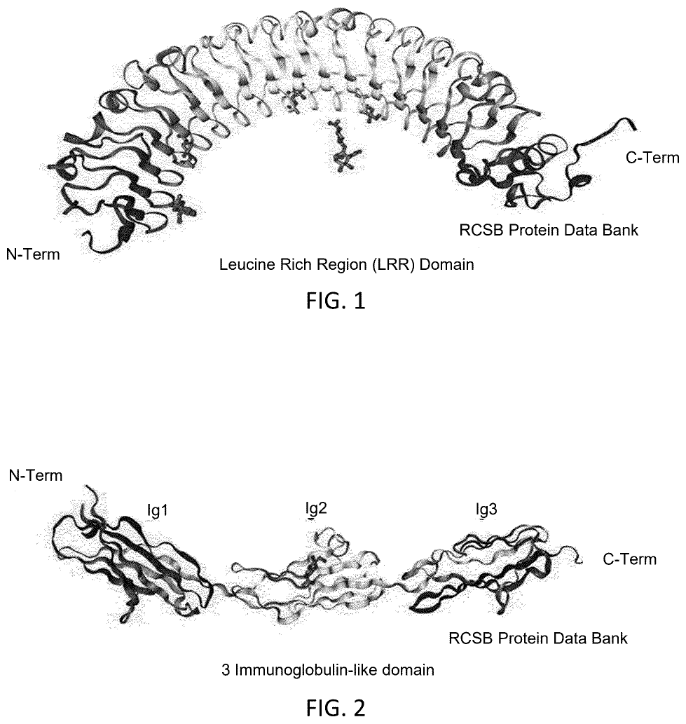

First, in order to predict the epitope by analyzing the three-dimensional steric structure of the extracellular domain (ECD) of the Lrig-1 protein, tools of Uniprot (http://www.uniprot.org) and RCSB Protein Data Bank (http://www.rcsb.org/pdb) were used to predict the three-dimensional steric structure, and the results are shown in FIGS. 1 and 2.

As shown in FIG. 1, a total of 15 leucine-rich regions (LRR1 to LRR15) were present in the Lrig-LRR domain (amino acid positions 41 to 494) in the extracellular domain of the Lrig-1 protein. Each of the LRR domains consisted of 23 to 27 amino acids and contained 3 to 5 leucine residues.

In addition, as shown in FIG. 2, three immunoglobulin-like domains were present at positions 494 to 781 in the amino acid sequence of the Lrig-1 protein in the extracellular domain of the Lrig-1 protein.

### [Example 2] Confirmation of Specific Expression of Lrig-1 mRNA in Regulatory T Cells

Verification was made as to whether the Lrig-1 protein could act as a biomarker specific for regulatory T cells.

For the verification, CD4⁺ T cells were isolated from mouse spleens by magnet-activated cell sorting (MACS) using CD4 beads. Subsequently, regulatory T (CD4⁺CD25⁺T) cells and non-regulatory T (CD4⁺CD25⁻T) cells were isolated by fluorescence-activated cell sorting (FACS) using CD25 antibody. For the isolated cells and the cells differentiated in Preparation Example 1, mRNA was extracted using Trizol, and then genomic DNA was removed from genomic RNA using a gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively analyze the expression level of Lrig-1 mRNA in the cDNA.

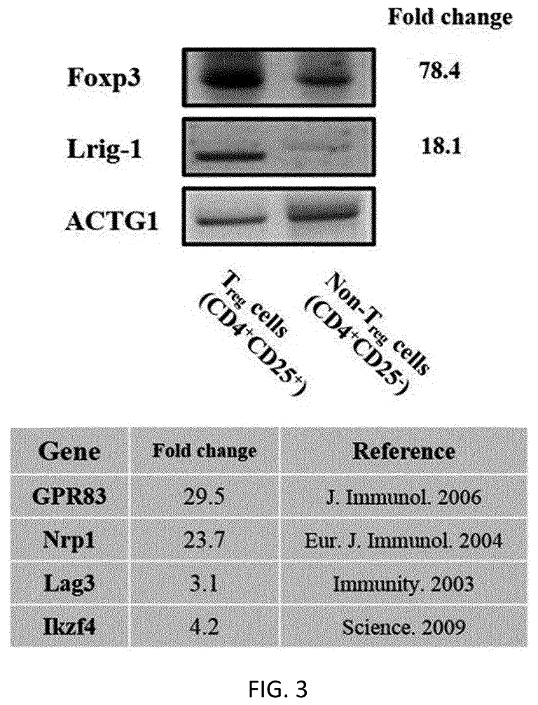

The real-time polymerase chain reaction was performed with primers shown in Table 2 below using SYBR Green (Molecular Probes) according to the protocol provided by the manufacturer for 40 cycles, each consisting of 95° C. for 3 minutes, 61° C. for 15 seconds, and 72° C. for 30 seconds. The relative gene expression level was calculated using the ΔCT method and normalized using HPRT. The results are shown in FIGS. 3 to 6.

TABLE 2

| Primer | Sequence |
|---|---|
| Mouse Lrig-1 | Forward 5′-GAC GGA ATT CAG TGA GGA GAA CCT-3′ |
| | Reverse 5′-CAA CTG GTA GTG GCA GCT TGT AGG-3′ |
| Mouse Lrig-2 | forward 5′-TCA CAA GGA ACA TTG TCT GAA CCA-3′ |
| | reverse 5′-GCC TGA TCT AAC ACA TCC TCC TCA-3′ |
| Mouse Lrig-3 | forward 5′-CAG CAC CTT GAG CTG AAC AGA AAC-3′ |
| | reverse 5′-CCA GCC TTT GGT AAT CTC GGT TAG-3′ |
| Mouse FOXP3 | forward 5′-CTT TCA CCT ATC CCA CCC TTA TCC-3′ |
| | reverse 5′-ATT CAT CTA CGG TCC ACA CTG CTC-3′ |
| ACTG1 | forward 5′-GGC GTC ATG GTG GGC ATG GG-3′ |
| | reverse 5′-ATG GCG TGG GGA AGG GCG TA-3′ |

As shown in FIG. 3, it can be seen that the expression level of Lrig-1 in the regulatory T (CD4⁺CD25⁺ T) cells was 18.1 times higher than that in the non-regulatory T (CD4⁺CD25⁻ T) cells. This was an about 10 times higher expression level than those of Lag3 and Ikzf4, which are previously known markers for regulatory T cells.

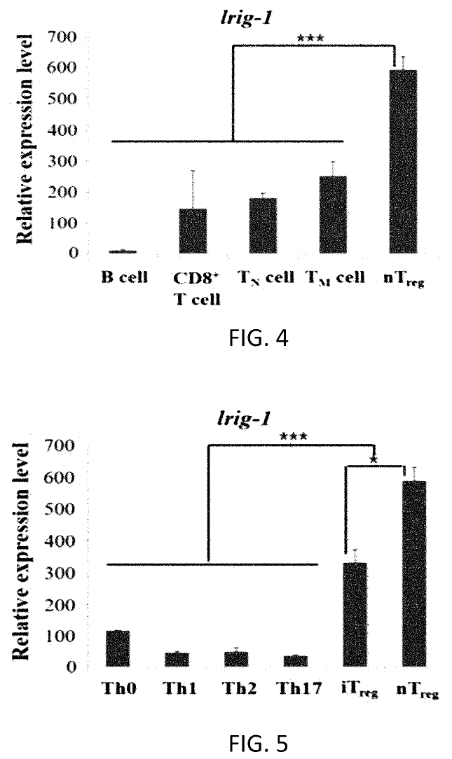

In addition, as shown in FIGS. 4 and 5, the expression level of Lrig-1 mRNA was remarkably high in the regulatory T cells compared to other types of immune cells, and in particular, was remarkably high in naturally isolated regulatory T cells (nTreg) compared to induced regulatory T cells (iTreg).

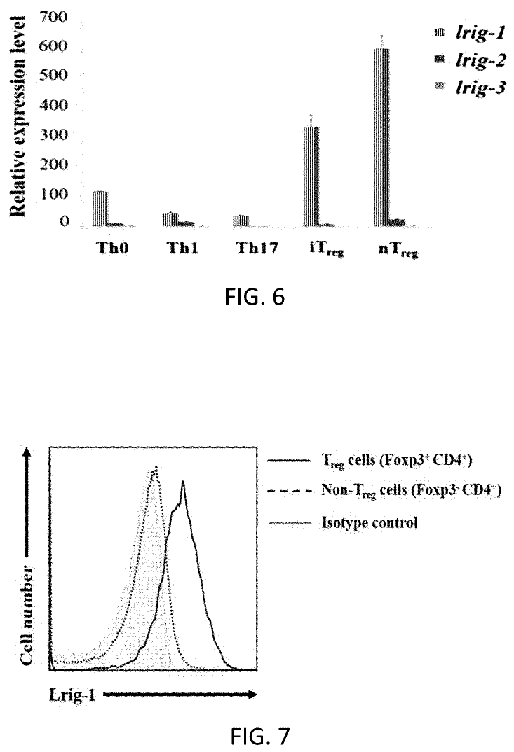

In addition, as shown in FIG. 6, the expression level of Lrig-1 was the highest among the expression levels of Lrig-1, Lrig-2 and Lrig-3 belonging to the Lrig family.

From the above results, it can be seen that the Lrig-1 protein according to the present invention is expressed specifically in regulatory T cells, in particular, naturally occurring regulatory T cells.

### [Example 3] Analysis of Specific Expression of Lrig-1 Protein in Regulatory T Cells

Analysis was performed as to whether the Lrig-1 protein expressed from Lrig-1 mRNA was specifically expressed only in regulatory T cells.

Using FOXP3-RFP-knocked-in mice having red fluorescence protein (RFP) conjugated to the FOXP3 promoter, which is a transcription factor specific for regulatory T cells, CD4⁺ T cells were isolated from the mouse spleens by magnet-activated cell sorting (MACS) using CD4 beads. Subsequently, regulatory T (CD4⁺RFP⁺ T) cells and non-regulatory T (CD4⁺RFP⁻ T) cells were isolated by fluorescence-activated cell sorting (FACS) using RFP protein. The isolated cells were stained with the purchased Lrig-1 antibody, a negative control was stained with an isotype antibody, and the expression levels of Lrig-1 were measured by fluorescence-activated cell sorting. The results are shown in FIG. 7.

As shown in FIG. 7, the non-regulatory T cells indicated by the dotted line showed almost the same Lrig-1 expression level as the negative control, whereas there were a large number of cells with a high Lrig-1 expression level in the regulatory T cells.

From the above results, it can be seen that the Lrig-1 protein according to the present invention is expressed specifically in regulatory T cells.

### [Example 4] Confirmation of Specific Expression of Lrig-1 Protein on Surface of Regulatory T Cells

Even when the Lrig-1 protein is expressed on the surfaces of regulatory T cells so that it becomes a target of cell therapy, the target therapy can be more effectively achieved. Accordingly, analysis was performed as to whether the Lrig-1 protein was expressed on the surfaces of the regulatory T cells.

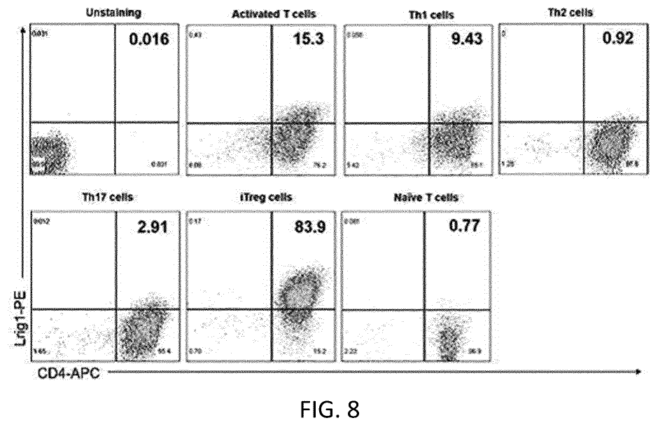

Each of the differentiated T cell subsets of Preparation Example 1 was stained with anti-CD4-APC and anti-Lrig-1-PE antibodies, and the expression levels of Lrig-1 on the surface of each subset was measured using fluorescence-activated cell sorting (FACS). The results are shown in FIG. 8.

As shown in FIG. 8, the expression levels of Lrig-1 in the activated T cells, Th1 cells, Th2 cells, Th17 cells, and naive T cells were 0.77 to 15.3, whereas the expression level of Lrig-1 in the differentiation-induced T cells (iTreg cells) was as high as 83.9.

From the above results, it can be seen that the Lrig-1 protein according to the present invention is not only expressed specifically in regulatory T (Treg) cells, but also expressed at a higher level, particularly on the surfaces of the Treg cells.

### [Production Examples 1 to 8] Production of Monoclonal Antibodies Specific for Lrig-1 Protein

Antibodies specific for the Lrig-1 protein according to the present invention were produced. The produced antibodies were antibodies capable of binding to any site on the Lrig-1 protein without being specified to a certain epitope.

In order to produce the antibodies, cells expressing the Lrig-1 protein were produced. More specifically, a DNA fragment corresponding to SEQ ID NO: 2 and pcDNA (hygro) were cleaved with a cleavage enzyme, incubated at 37° C., and ligated to each other to produce pcDNA into which the DNA sequence of the Lrig-1 protein has been inserted. The produced pcDNA into which SEQ ID NO: 2 has been inserted was introduced into L cells through transfection so that the Lrig-1 protein could be expressed on the surfaces of the L cells.

Light and heavy chain amino acid sequences capable of binding to the Lrig-1 protein expressed on the cell surfaces were selected from the Human scFv library, thereby selecting a total of eight heavy and light chains.

Monoclonal antibodies were produced by fusing the selected heavy and light chain amino acid sequences with the mlgG2a Fc region or the human IgG1 Fc region. The sequences of the monoclonal antibodies are shown in Table 3 below.

TABLE 3

| | | | | Sequence |
|---|---|---|---|---|
| | Clone | Position | AminQ acid sequence | information |
| Production | GTC210-01 | Heavy | EVQLLESGGG LVQPGGSLRL SCAASGFTFS | SEQ ID NO: |
| Example 1 | clone | chain | GYDMSWVRQA PGKGLEWVSL IYPDSGNKYY 60 | 147 |
| | | | ADSVKGRFTI SRDNSKNTLY LQMNSLRAED | |
| | | | TAVYYCARDA GLSWAGAFDY WGQGTLVTVS 120 | |
| | | | SASTTAPSVY PLAPVCGDTT GSSVTLGCLV | |
| | | | KGYFPEPVTL TWNSGSLSSG VHTFPAVLQS 180 | |
| | | | DLYTLSSSVT VTSSTWPSQS ITCNVAHPAS | |
| | | | STKVDKKIEP RGPTIKPCPP CKCPAPNLLG 240 | |
| | | | GPSVFIFPPK IKDVLMISLS PIVTCVVVDV | |
| | | | SEDDPDVQIS WFVNNVEVHT AQTQTHREDY 300 | |
| | | | NSTLRVVSAL PIQHQDWMSG KEFKCKVNNK | |
| | | | DLPAPIERTI SKPKGSVRAP QVYVLPPPEE 360 | |
| | | | EMTKKQVTLT CMVTDFMPED IYVEWTNNGK | |
| | | | TELNYKNTEP VLDSDGSYFM YSKLRVEKKN 420 | |
| | | | WVERNSYSCS VVHEGLHNHH TTKSFSRTPG K | |
| | | Light | QSVLTQPPSA SGTPGQRVTI SCSGSSSNIG | SEQ ID NO: |
| | | chain | SNYVTWYQQL PGTAPKLLIY SDSHRPSGVP 60 | 148 |
| | | | DRFSGSKSGT SASLAISGLQ SEDEADYYCG | |
| | | | SWDYSLSAYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC210-02 | Heavy | EVQLLESGGG LVQPGGSLRL SCAASGFTFS | SEQ ID NO: |
| Example 2 | clone | chain | NYYMSWVRQA PGKGLEWVSG ISPGDSSTYY 60 | 149 |
| | | | ADSVKGRFTI SRDNSKNTLY LQMNSLRAED | |
| | | | TAVYYCAKGL YSNPNEPFDY WGQGTLVTVS 120 | |
| | | | SASTTAPSVY PLAPVCGDTT GSSVTLGCLV | |
| | | | KGYFPEPVTL TWNSGSLSSG VHTFPAVLQS 180 | |
| | | | DLYTLSSSVT VTSSTWPSQS ITCNVAHPAS | |
| | | | STKVDKKIEP RGPTIKPCPP CKCPAPNLLG 240 | |
| | | | GPSVFIFPPK IKDVLMISLS PIVTCVVVDV | |
| | | | SEDDPDVQIS WFVNNVEVHT AQTQTHREDY 300 | |
| | | | NSTLRVVSAL PIQHQDWMSG KEFKCKVNNK | |
| | | | DLPAPIERTI SKPKGSVRAP QVYVLPPPEE 360 | |
| | | | EMTKKQVTLT CMVTDFMPED IYVEWTNNGK | |
| | | | TELNYKNTEP VLDSDGSYFM YSKLRVEKKN 420 | |
| | | | WVERNSYSCS VVHEGLHNHH TTKSFSRTPG K | |
| | | Light | QSVLTQPPSA SGTPGQRVTI SCTGSSSNIG | SEQ ID NO: |
| | | chain | SNYVSWYQQL PGTAPKLLIY DDSQRPSGVP 60 | 150 |
| | | | DRFSGSKSGT SASLAISGLR SEDEADYYCG | |
| | | | TWDYSLNGYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC210-03 | Heavy | EVQLLESGGG LVQPGGSLRL SCAASGFTFS | SEQ ID NO: |
| Example 3 | clone | chain | SYDMSWVRQA PGKGLEWVSG ISPDGSNIYY 60 | 151 |
| | | | ADSVKGRFTI SRDNSKNTLY LQMNSLRAED | |
| | | | TAVYYCAKVG LRCRYEACSY AYGMDVWGQG 120 | |
| | | | TLVTVSSAST TAPSVYPLAP VCGDTTGSSV | |
| | | | TLGCLVKGYF PEPVTLTWNS GSLSSGVHTF 180 | |
| | | | PAVLQSDLYT LSSSVTVTSS TWPSQSITCN | |
| | | | VAHPASSTKV DKKIEPRGPT IKPCPPCKCP 240 | |
| | | | APNLLGGPSV FIFPPKIKDV LMISLSPIVT | |
| | | | CVVVDVSEDD PDVQISWFVN NVEVHTAQTQ 300 | |
| | | | THREDYNSTL RVVSALPIQH QDWMSGKEFK | |
| | | | CKVNNKDLPA PIERTISKPK GSVRAPQVYV 360 | |
| | | | LPPPEEEMTK KQVTLTCMVT DFMPEDIYVE | |
| | | | WTNNGKTELN YKNTEPVLDS DGSYFMYSKL 420 | |
| | | | RVEKKNWVER NSYSCSVVHE GLHNHHTTKS | |
| | | | FSRTPGK | |
| | | Light | QSVLTQPPSA SGTPGQRVTI SCSGSSSNIG | SEQ ID NO: |
| | | chain | SNYVSWYQQL PGTAPKLLIY SDSHRPSGVP 60 | 152 |
| | | | DRFSGSKSGT SASLAISGLR SEDEADYYCA | |
| | | | TWDSSLNGYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC 210-04 | Heavy | EVQLLESGGG LVQPGGSLRL SCAASGFTFS | SEQ ID NO: |
| Example 4 | clone | chain | NYDMSWVRQA PGKGLEWVSS ISPSSGSIYY 60 | 153 |
| | | | ADSVKGRFTI SRDNSKNTLY LQMNSLRAED | |
| | | | TAVYYCAKDL DAFWRPSFDY WGQGTLVTVS 120 | |
| | | | STTAPSVYPL APVCGDTTGS SVTLGCLVKG | |
| | | | YFPEPVTLTW NSGSLSSGVH TFPAVLQSDL 180 | |
| | | | YTLSSSVTVT SSTWPSQSIT CNVAHPASST | |
| | | | KVDKKIEPRG PTIKPCPPCK CPAPNLLGGP 240 | |
| | | | SVFIFPPKIK DVLMISLSPI VTCVVVDVSE | |
| | | | DDPDVQISWF VNNVEVHTAQ TQTHREDYNS 300 | |
| | | | TLRVVSALPI QHQDWMSGKE FKCKVNNKDL | |
| | | | PAPIERTISK PKGSVRAPQV YVLPPPEEEM 360 | |
| | | | TKKQVTLTCM VTDFMPEDIY VEWTNNGKTE | |
| | | | LNYKNTEPVL DSDGSYFMYS KLRVEKKNWV 420 | |
| | | | ERNSYSCSVV HEGLHNHHTT KSFSRTPGK | |
| | | Light | QSVLTQPPSA SGTPGQRVTI SCTGSSSNIG | SEQ ID NO: |
| | | chain | NNNVNWYQQL PGTAPKLLIY SDSHRPSGVP 60 | 154 |
| | | | DRFSGSKSGT SASLAISGLR SEDEADYYCG | |
| | | | SWDDSLSAYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC110-01 | Heavy | EVQLLESGGG LVQPGGSLRL SCAASGFTFS | SEQ ID NO: |
| Example 5 | clone | chain | NYDMSWVRQA PGKGLEWVSS ISPSSGSIYY 60 | 155 |
| | | | ADSVKGRFTI SRDNSKNTLY LQMNSLRAED | |
| | | | TAVYYCAKDL DAFWRPSFDY WGQGTLVTVS 120 | |
| | | | SASTTAPSVY PLAPVCGDTT GSSVTLGCLV | |
| | | | KGYFPEPVTL TWNSGSLSSG VHTFPAVLQS 180 | |
| | | | DLYTLSSSVT VTSSTWPSQS ITCNVAHPAS | |
| | | | STKVDKKIEP RGPTIKPCPP CKCPAPNLLG 240 | |
| | | | GPSVFIFPPK IKDVLMISLS PIVTCVVVDV | |
| | | | SEDDPDVQIS WFVNNVEVHT AQTQTHREDY 300 | |
| | | | NSTLRVVSAL PIQHQDWMSG KEFKCKVNNK | |
| | | | DLPAPIERTI SKPKGSVRAP QVYVLPPPEE 360 | |
| | | | EMTKKQVTLT CMVTDFMPED IYVEWTNNGK | |
| | | | TELNYKNTEP VLDSDGSYFM YSKLRVEKKN 420 | |
| | | | WVERNSYSCS VVHEGLHNHH TTKSFSRTPG K | |
| | | Light | QSVLTQPPSA SGTPGQRVTI SCTGSSSNIG | SEQ ID NO: |
| | | chain | NNSVTWYQQL PGTAPKLLIY ADNNRPSGVP 60 | 156 |
| | | | DRFSGSKSGT SASLAISGLR SEDEADYYCA | |
| | | | AWDSSLSAYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC110-02 | Heavy | EVQLLESGGG LVQPGGSLRL SCAASGFTFS | SEQ ID NO: |
| Example 6 | clone | chain | DYYMSWVRQA PGKGLEWVSG ISHDSGSKYY 60 | 157 |
| | | | ADSVKGRFTI SRDNSKNTLY LQMNSLRAED | |
| | | | TAVYYCARHW TTFDYWGQGT LVTVSSASTT 120 | |
| | | | APSVYPLAPV CGDTTGSSVT LGCLVKGYFP | |
| | | | EPVTLTWNSG SLSSGVHTFP AVLQSDLYTL 180 | |
| | | | SSSVTVTSST WPSQSITCNV AHPASSTKVD | |
| | | | KKIEPRGPTI KPCPPCKCPA PNLLGGPSVF 240 | |
| | | | IFPPKIKDVL MISLSPIVTC VVVDVSEDDP | |
| | | | DVQISWFVNN VEVHTAQTQT HREDYNSTLR 300 | |
| | | | VVSALPIQHQ DWMSGKEFKC KVNNKDLPAP | |
| | | | IERTISKPKG SVRAPQVYVL PPPEEEMTKK 360 | |
| | | | QVTLTCMVTD FMPEDIYVEW TNNGKTELNY | |
| | | | KNTEPVLDSD GSYFMYSKLR VEKKNWVERN 420 | |
| | | | SYSCSVVHEG LHNHHTTKSF SRTPGK | |
| | | Light | QSVLTQPPSA SGTPGQRVTI SCSGSSSNIG | SEQ ID NO: |
| | | chain | SNNVTWYQQL PGTAPKLLIY ANSNRPSGVP 60 | 158 |
| | | | DRFSGSKSGT SASLAISGLR SEDEADYYCG | |
| | | | AWDYSLSAYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC110-03 | Heavy | EVQLLESGGG LVQPGGSLRL SCAASGFTFS | SEQ ID NO: |
| Example 7 | clone | chain | NYAMSWVRQA PGKGLEWVSA IYPGGGSIYY 60 | 159 |
| | | | ADSVKGRFTI SRDNSKNTLY LQMNSLRAED | |
| | | | TAVYYCARDI LPCPWGRCYY DYAMDVWGQG 120 | |
| | | | TLVTVSSAST TAPSVYPLAP VCGDTTGSSV | |
| | | | TLGCLVKGYF PEPVTLTWNS GSLSSGVHTF 180 | |
| | | | PAVLQSDLYT LSSSVTVTSS TWPSQSITCN | |
| | | | VAHPASSTKV DKKIEPRGPT IKPCPPCKCP 240 | |
| | | | APNLLGGPSV FIFPPKIKDV LMISLSPIVT | |
| | | | CVVVDVSEDD PDVQISWFVN NVEVHTAQTQ 300 | |
| | | | THREDYNSTL RVVSALPIQH QDWMSGKEFK | |
| | | | CKVNNKDLPA PIERTISKPK GSVRAPQVYV 360 | |
| | | | LPPPEEEMTK KQVTLTCMVT DFMPEDIYVE | |
| | | | WTNNGKTELN YKNTEPVLDS DGSYFMYSKL 420 | |
| | | | RVEKKNWVER NSYSCSVVHE GLHNHHTTKS | |
| | | | FSRTPGK | |
| | | Light | QSVLTQPPSA SGTPGQRVTI SCSDSSSNIG | SEQ ID NO: |
| | | chain | SNTVSWYQQL PGTAPKLLIY ADNNRPSGVP 60 | 160 |
| | | | DRFSGSKSGT SASLAISGLR SEDEADYYCG | |
| | | | TWDYSLSGYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC110-04 | Heavy | EVQLLESGGG LVQPGGSLRL SCAASGFTFS | SEQ ID NO: |
| Example 8 | clone | chain | NYAMSWVRQA PGKGLEWVSV ISHGGGSTYY 60 | 161 |
| | | | ADSVKGRFTI SRDNSKNTLY LQMNSLRAED | |
| | | | TAVYYCARVI SNCHLGVCYY SNGMDVWGQG 120 | |
| | | | TLVTVSSAST TAPSVYPLAP VCGDTTGSSV | |
| | | | TLGCLVKGYF PEPVTLTWNS GSLSSGVHTF 180 | |
| | | | PAVLQSDLYT LSSSVTVTSS TWPSQSITCN | |
| | | | VAHPASSTKV DKKIEPRGPT IKPCPPCKCP 240 | |
| | | | APNLLGGPSV FIFPPKIKDV LMISLSPIVT | |
| | | | CVVVDVSEDD PDVQISWFVN NVEVHTAQTQ 300 | |
| | | | THREDYNSTL RVVSALPIQH QDWMSGKEFK | |
| | | | CKVNNKDLPA PIERTISKPK GSVRAPQVYV 360 | |
| | | | LPPPEEEMTK KQVTLTCMVT DFMPEDIYVE | |
| | | | WTNNGKTELN YKNTEPVLDS DGSYFMYSKL 420 | |
| | | | RVEKKNWVER NSYSCSVVHE GLHNHHTTKS | |
| | | | FSRTPGK | |
| | | Light | QSVLTQPPSA SGTPGQRVTI SCSGSSSNIG | SEQ ID NO: |
| | | chain | NNDVYWYQQL PGTAPKLLIY SDSQRPSGVP 60 | 162 |
| | | | DRFSGSKSGT SASLAISGLR SEDEADYYCG | |
| | | | TWDYSLSGYV FGGGTKLTVL RTVAAPTVSI 120 | |
| | | | FPPSSEQLTS GGASVVCFLN NFYPKDINVK | |
| | | | WKIDGSERQN GVLNSWTDQD SKDSTYSMSS 180 | |
| | | | TLTLTKDEYE RHNSYTCEAT HKTSTSPIVK | |
| | | | SFNRNEC | |
| Production | GTC110-04 | Heavy | EVQLLESGGG LVQPGGSLRL SCAASGFTFS | SEQ ID NO: |
| Example 9 | humanized | chain | NYAMSWVRQA PGKGLEWVSV ISHGGGSTYY 60 | 163 |
| | antibody | | ADSVKGRFTI SRDNSKNTLY LQMNSLRAED | |
| | | | TAVYYCARVI SNCHLGVCYY SNGMDVWGQG 120 | |
| | | | TLVTVSSAST KGPSVFPLAP SSKSTSGGTA | |
| | | | ALGCLVKDYF PEPVTVSWNS GALTSGVHTF 180 | |
| | | | PAVLQSSGLY SLSSVVTVPS SSLGTQTYIC | |
| | | | NVNHKPSNTK VDKKVEPKSC DKTHTCPPCP 240 | |
| | | | APELLGGPSV FLFPPKPKDT LMISRTPEVT | |
| | | | CVVVDVSHED PEVKFNWYVD GVEVHNAKTK 300 | |
| | | | PREEQYNSTY RVVSVLTVLH QDWLNGKEYK | |
| | | | CKVSNKALPA PIEKTISKAK GQPREPQVYT 360 | |
| | | | LPPSREEMTK NQVSLTCLVK GFYPSDIAVE | |
| | | | WESNGQPENN YKTTPPVLDS DGSFFLYSKL 420 | |
| | | | TVDKSRWQQG NVFSCSVMHE ALHNHYTQKS | |
| | | | LSLSPGKY | |
| | | Light | QSVLTQPPSA SGTPGQRVTI SCSGSSSNIG | SEQ ID NO: |
| | | chain | NNDVYWYQQL PGTAPKLLIY SDSQRPSGVP 60 | 164 |
| | | | DRFSGSKSGT SASLAISGLR SEDEADYYCG | |
| | | | TWDYSLSGYV FGGGTKLTVL RTVAAPSVFI 120 | |
| | | | FPPSDEQLKS GTASVVCLLN NFYPREAKVQ | |
| | | | WKVDNALQSG NSQESVTEQD SKDSTYSLSS 180 | |
| | | | TLTLSKADYE KHKVYACEVT HQGLSSPVTK | |
| | | | SFNRGEC | |

### [Example 5] Evaluation of Binding Affinities of Antibodies According to the Present Invention for Lrig-1 Protein

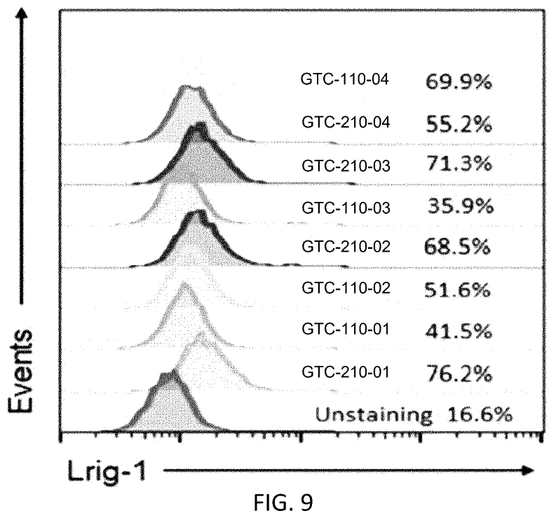

In order to examine whether the monoclonal antibodies according to the present invention, produced in Production Examples 1 to 8, well recognize Lrig-1, each of the antibodies of Production Examples 1 to 8 was bound to L cells that stably express Lrig-1, and then an eFlour 670-conjugated secondary antibody capable of recognizing mouse antibodies was added thereto. Next, the binding affinities of the monoclonal antibodies for the Lrig-1 protein were analyzed using FACS. The results are shown in FIG. 9.

As shown in FIG. 9, it could be confirmed that all the Lrig-1 protein-specific monoclonal antibodies according to the present invention effectively recognized and bound to the Lrig-1 protein present on the surfaces of the L cells.

### [Example 6] Regulation of Signaling Pathway in Regulatory T Cells by Antibodies According to the Present Invention

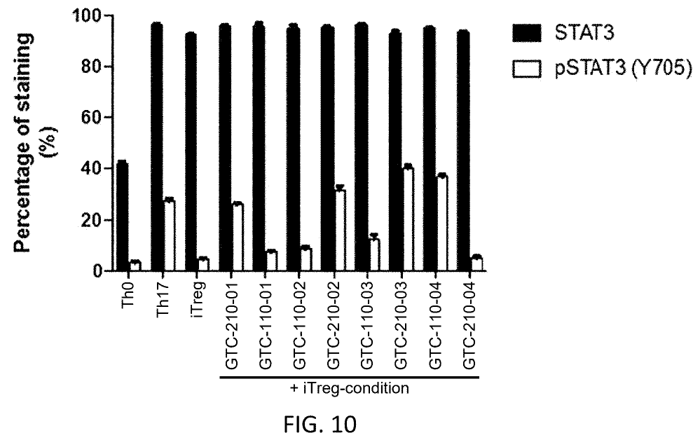

In order to analyze how the monoclonal antibodies according to the present invention, produced in Production Examples 1 to 8, affect the signaling pathway in regulatory T cells through the Lrig-1 protein, Lrig-1 present on the surfaces of the regulatory T cells was stimulated by treating the regulatory T cells with the antibodies of Production Examples 1 to 8, and then the tyrosine phosphorylation level of Stat3 protein present in the stimulated regulatory T cells was analyzed by phosphotyrosine immunoblot assay. The results are shown in FIG. 10.

As shown in FIG. 10, it could be confirmed that the Lrig-1 protein-specific monoclonal antibodies (GTC210-01, GTC210-02, GTC210-03 and GTC210-04) according to the present invention increased phosphorylation of Stat3 to the same level as that in Th17 cells. Meanwhile, it could be confirmed that the Lrig-1 protein-specific monoclonal antibodies (GTC110-01, GTC110-02, GTC110-03 and GTC110-04) according to the present invention continued to maintain and decrease the phosphorylation of Stat3 at the same level as that in iTreg cells.

### [Example 7] Cancer Therapeutic Effects of GTC110-01, GTC110-02, GTC110-03 and GTC110-04 Antibodies

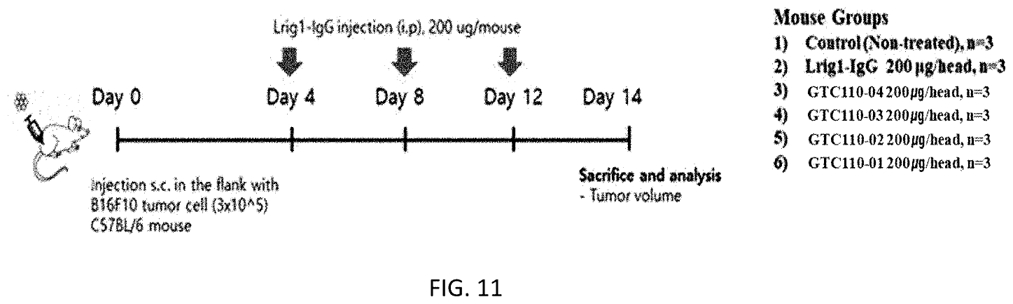

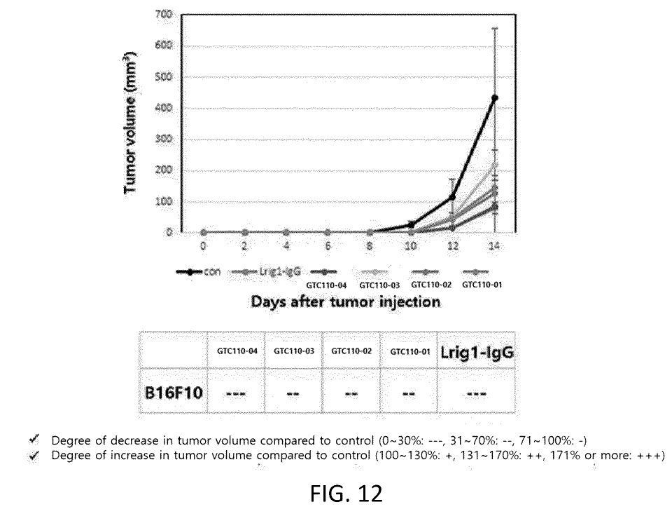

In order to evaluate the solid cancer therapeutic effects of the monoclonal antibodies (GTC110-01, GTC110-02, GTC110-03 and GTC110-04) according to the present invention, produced in Production Examples 5 to 8, as shown in FIG. 11, B16F10 melanoma cells were subcutaneously injected into the dorsal area of mice in an amount of 3×10⁵ cells, and then each of the antibodies of Production Examples 5 to 8 was intraperitoneally injected into the mice in an amount of 200 ug on days 4, 8, and 12. After transplantation of the melanoma cells, time-dependent changes in the tumor volume were measured, and the results of the measurement are shown in FIG. 12.

As shown in FIG. 12, it could be confirmed that the tumor volume remarkably decreased when the cells were treated with the Lrig-1 protein-specific monoclonal antibodies (GTC110-01, GTC110-02, GTC110-03 and GTC110-04) according to the present invention, compared to the negative control group which was not treated with the antibody.

Thereby, it can be seen that the Lrig-1 protein-specific monoclonal antibodies according to the present invention can inhibit the growth of various solid cancer cells, thereby effectively preventing, ameliorating or treating such cancers.

### [Example 8] Identification of Epitope of Lrig-1 Protein According to the Present Invention

In order to discover a conformational epitope, to which the GTC110-04 antibody of Production Example 8 can bind, in the extracellular domain of the Lrig-1 protein, the following experiment was conducted.

### [8-1] Covalent Labeling Mass Spectroscopy (MS) (CL-MS)

### [8-1-1] Epitope Identification Using Chymotrypsin

An antigen-antibody conjugate was prepared by mixing the GTTC110-04 antibody of Preparation Example 8 and the extracellular domain (antigen) of Lrig-1 protein represented by SEQ ID NO: 4 at a ratio of 2:1 and sufficiently reacting the mixture, and the non-antibody-conjugated extracellular domain (antigen) of Lrig-1 protein was also prepared. Next, DEPC (diethyl pyrocarbonate) was added to each of the samples so as not to exceed 1%, and then allowed to react sufficiently at 37° C. for 1 minute, and the reaction was completed by adding imidazole. After completion of the reaction, methanol was added to each of the samples, and the supernatant was removed by centrifugation. Thereafter, each sample was resuspended in a solution containing 8 M urea and 0.1 M NaCl, and then chymotrypsin was added thereto so that the peptide bond could be broken.

Each of the samples in which the peptide bond was broken as described above was subjected to fluid chromatography (Vanquish™ Flex Quaternary UHPLC (Thermo SCIENTIFIC)) using a Hypersil GOLD™ (1.9 μm, 1×100 mm) column at 30° C. under the conditions shown in Table 4 below.

TABLE 4

| | Total time | Flow rate | Mobile phase | Mobile phase |
|---|---|---|---|---|
| Step | (min) | (μl/min) | A* (%) | B** (%) |
| 0 | 0.00 | 100.00 | 95.0 | 5.0 |
| 1 | 5.00 | 100.00 | 95.0 | 5.0 |
| 2 | 7.00 | 100.00 | 90.0 | 10.0 |
| 3 | 48.00 | 100.00 | 70.0 | 30.0 |
| 4 | 55.00 | 100.00 | 5.0 | 95.0 |
| 5 | 65.00 | 100.00 | 5.0 | 95.0 |
| 6 | 66.00 | 100.00 | 95.00 | 5.0 |
| 7 | 71.00 | 100.00 | 95.00 | 5.0 |

*mobile phase A (distilled water/0.2% formic acid)
**mobile phase B (acetonitrile/0.2% formic acid)

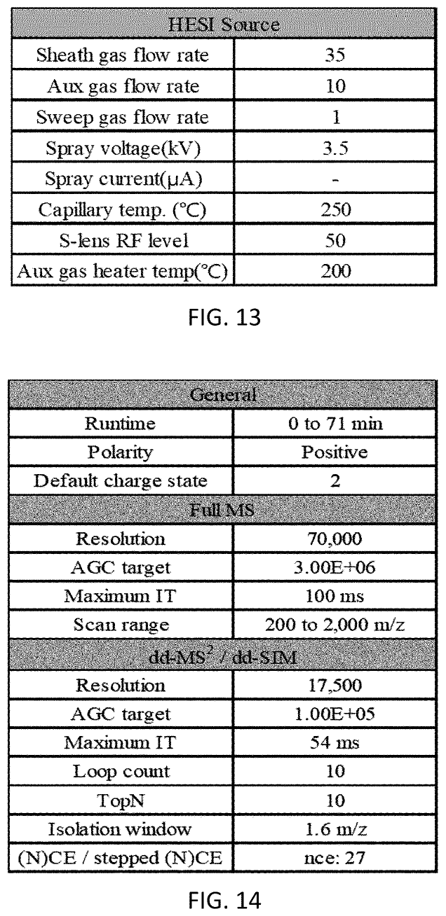

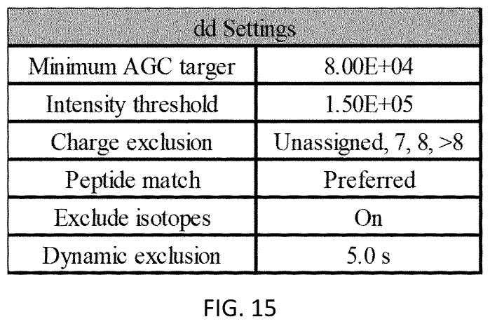

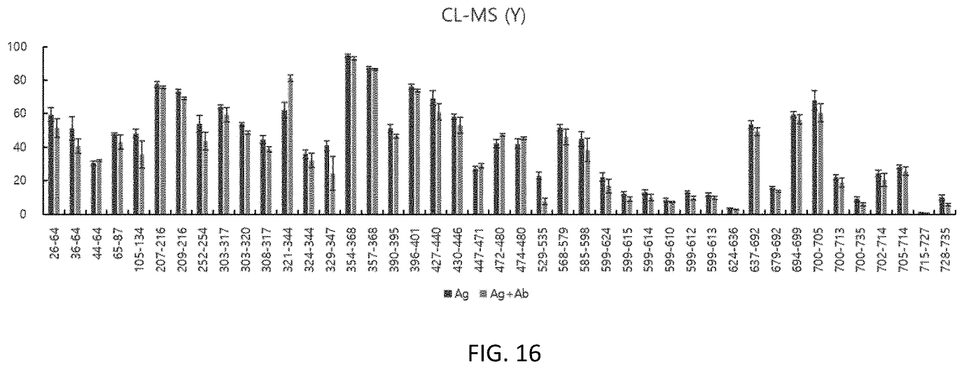

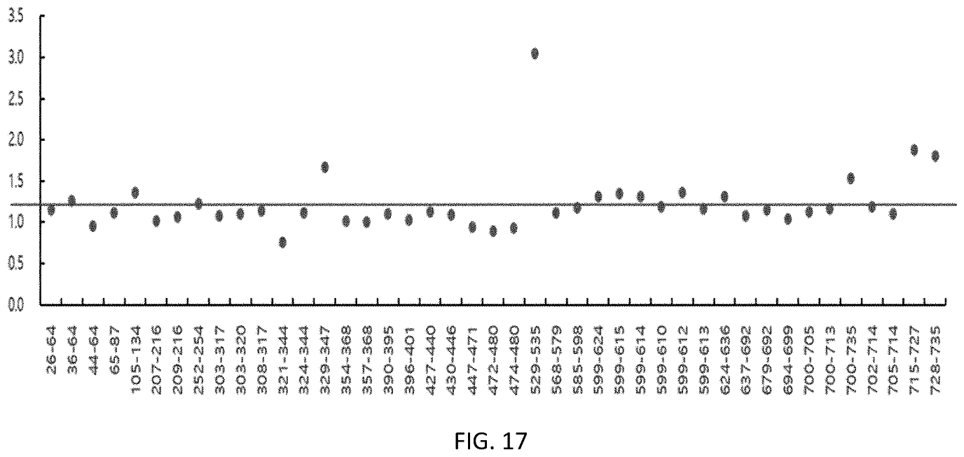

Thereafter, mass spectrometry (Q Exactive™ Plus Mass Spectrometer (Thermo SCIENTIFIC)) was performed under the conditions shown in FIGS. 13 to 15, and the measured mass spectrometry values of the antigen-antibody conjugate and the antigen were compared, and the results were shown in FIG. 16 and Table 5 below. Furthermore, from the measured mass spectrometry values, the ratio of the antigen-antibody conjugate/the antigen was calculated, and the results are shown in FIG. 17.

TABLE 5

| | | | Antigen- | | | | |
|---|---|---|---|---|---|---|---|
| Amino | | | antibody | | | | |
| acid | Antigen | | conjugate | | | | |
| positions | Mean | S.D | mean | S.D | Difference | Ratio | p value |
| 26-64 | 59.56 | 4.11 | 51.51 | 5.56 | 8.05 | 1.16 | ** |
| 36-64 | 51.37 | 7.00 | 40.77 | 4.25 | 10.60 | 1.26 | ** |
| 44-64 | 30.46 | 1.29 | 31.87 | 0.66 | −1.41 | 0.96 | * |
| 65-87 | 47.90 | 0.74 | 43.09 | 4.36 | 4.81 | 1.11 | * |
| 105-134 | 48.26 | 2.60 | 35.54 | 8.20 | 12.72 | 1.36 | ** |
| 207-216 | 77.52 | 1.61 | 75.76 | 0.72 | 1.77 | 1.02 | * |
| 209-216 | 73.44 | 1.19 | 169.09 | 0.63 | 4.34 | 1.06 | *** |
| 252-254 | 53.84 | 5.08 | 43.75 | 5.22 | 10.09 | 1.23 | ** |
| 303-317 | 64.17 | 1.22 | 59.52 | 4.26 | 4.65 | 1.08 | * |
| 303-320 | 53.78 | 0.94 | 48.69 | 0.82 | 5.10 | 1.10 | *** |
| 308-317 | 44.52 | 2.44 | 38.84 | 1.67 | 5.68 | 1.15 | *** |
| 321-344 | 62.11 | 4.53 | 81.23 | 1.84 | −19.12 | 0.76 | *** |
| 324-344 | 35.87 | 2.41 | 32.23 | 4.19 | 3.64 | 1.11 | * |
| 329-347 | 40.96 | 2.95 | 24.52 | 10.14 | 16.44 | 1.67 | ** |
| 354-368 | 94.66 | 0.98 | 92.95 | 1.02 | 1.71 | 1.02 | ** |
| 357-368 | 87.35 | 0.80 | 86.48 | 0.59 | 0.88 | 1.01 | * |
| 390-395 | 51.34 | 2.27 | 46.62 | 1.05 | 4.72 | 1.10 | *** |
| 396-401 | 76.00 | 1.59 | 73.84 | 0.93 | 2.15 | 1.03 | ** |
| 427-440 | 69.31 | 4.49 | 61.15 | 4.78 | 8.16 | 1.13 | ** |
| 430-446 | 58.32 | 1.57 | 53.15 | 4.67 | 5.17 | 1.10 | * |
| 447-471 | 27.21 | 1.42 | 28.88 | 1.33 | −1.67 | 0.94 | * |
| 472-480 | 42.21 | 2.48 | 47.49 | 0.82 | −5.28 | 0.89 | *** |
| 474-480 | 42.02 | 3.00 | 45.34 | 0.87 | −3.32 | 0.93 | * |
| 529-535 | 22.96 | 2.26 | 7.52 | 2.03 | 15.44 | 3.05 | *** |
| 568-579 | 51.68 | 2.04 | 46.19 | 4.75 | 5.49 | 1.12 | ** |
| 585-598 | 44.89 | 4.28 | 38.20 | 7.01 | 6.69 | 1.18 | * |
| 599-624 | 22.09 | 2.48 | 16.84 | 4.15 | 5.25 | 1.31 | ** |
| 599-615 | 12.33 | 1.08 | 9.13 | 1.40 | 3.20 | 1.35 | *** |
| 599-614 | 13.25 | 1.26 | 10.06 | 2.00 | 3.19 | 1.32 | ** |
| 599-610 | 8.54 | 1.11 | 7.16 | 0.49 | 1.38 | 1.19 | ** |
| 599-612 | 12.89 | 0.77 | 9.49 | 1.18 | 3.39 | 1.36 | *** |
| 599-613 | 11.49 | 1.32 | 9.85 | 0.94 | 1.65 | 1.17 | ** |
| 624-636 | 3.40 | 0.20 | 2.58 | 0.21 | 0.82 | 1.32 | *** |
| 637-692 | 53.44 | 2.57 | 49.35 | 2.29 | 4.09 | 1.08 | ** |
| 679-692 | 15.77 | 0.70 | 13.62 | 0.74 | 2.15 | 1.16 | *** |
| 694-699 | 59.44 | 1.75 | 56.79 | 2.66 | 2.65 | 1.05 | * |
| 700-705 | 67.98 | 5.79 | 60.47 | 5.42 | 7.51 | 1.12 | * |
| 700-713 | 21.98 | 1.53 | 18.80 | 2.73 | 3.18 | 1.17 | * |
| 700-735 | 19.04 | 1.23 | 5.87 | 1.02 | 3.17 | 1.54 | *** |
| 702-714 | 24.37 | 2.05 | 20.49 | 3.86 | 3.88 | 1.19 | * |
| 705-714 | 28.28 | 1.17 | 25.69 | 2.46 | 2.59 | 1.10 | * |
| 715-727 | 0.66 | 0.26 | 0.35 | 0.11 | 0.31 | 1.88 | ** |
| 728-735 | 10.05 | 1.56 | 5.58 | 0.81 | 4.47 | 1.80 | *** |

As shown in FIGS. 16 and 17 and Table 5, it was confirmed that the polypeptides consisting of the amino acids at positions 36 to 64, 105 to 134, 252 to 254, 329 to 347, 529 to 535, 599 to 612, 599 to 614, 599 to 615, 599 to 624, 624 to 636, 700 to 735, 715 to 727, and 728 to 735, respectively, in the extracellular domain of Lrig-1 protein represented by SEQ ID NO: 4, had an antigen-antibody conjugate/antigen ratio of 1.2 or more, suggesting that these polypeptides were likely to be selected as epitopes of the Lrig-1 protein. Furthermore, it was confirmed that the polypeptides consisting of the amino acid sequences represented by SEQ ID NOs: 7 to 13 showed a more significant actual difference value, and thus were more likely to be selected as epitopes.

### [8-1-2] Epitope Identification Using Trypsin

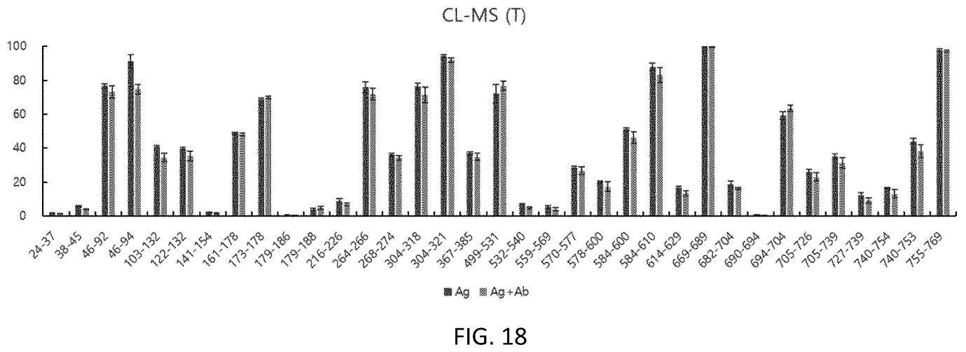

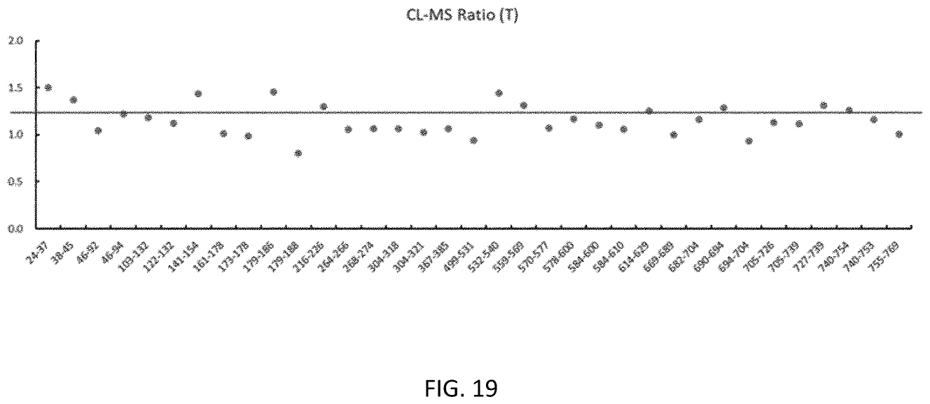

An experiment for epitope identification was conducted in the same manner as described in [8-1-1] above, except that trypsin was used instead of chymotrypsin as the enzyme. The results are shown in FIGS. 18 and 19 and Table 6 below.

TABLE 6

| | | | Antigen- | | | | |
|---|---|---|---|---|---|---|---|
| Amino | | | antibody | | | | |
| acid | Antigen | | conjugate | | | | |
| positions | Mean | S.D | mean | S.D | difference | Ratio | p value |
| 24-37 | 1.67 | 0.34 | 1.11 | 0.19 | 0.56 | 1.51 | ** |
| 38-45 | 5.70 | 0.33 | 4.15 | 0.15 | 1.55 | 1.37 | *** |
| 46-92 | 76.64 | 1.30 | 73.22 | 3.67 | 3.42 | 1.05 | * |
| 46-94 | 91.22 | 3.99 | 74.75 | 2.92 | 16.47 | 1.22 | *** |
| 103-132 | 40.74 | 1.01 | 34.52 | 2.64 | 6.23 | 1.18 | *** |
| 122-132 | 39.75 | 0.85 | 35.32 | 2.70 | 4.43 | 1.13 | ** |
| 141-154 | 1.99 | 0.42 | 1.38 | 0.35 | 0.61 | 1.44 | ** |
| 161-178 | 48.76 | 0.58 | 48.00 | 0.75 | 0.75 | 1.02 | * |
| 173-178 | 68.68 | 0.96 | 69.78 | 0.72 | −1.10 | 0.98 | * |
| 179-186 | 0.45 | 0.09 | 0.31 | 0.08 | 0.14 | 1.46 | ** |
| 179-188 | 3.91 | 0.71 | 4.87 | 0.97 | −0.96 | 0.80 | * |
| 216-226 | 8.82 | 1.60 | 6.80 | 0.93 | 2.03 | 1.30 | ** |
| 264-266 | 75.97 | 3.22 | 71.79 | 3.45 | 4.18 | 1.06 | * |
| 268−274 | 36.31 | 0.82 | 34.17 | 1.44 | 2.14 | 1.06 | ** |
| 304-318 | 76.26 | 2.16 | 71.49 | 4.45 | 4.77 | 1.07 | * |
| 304-321 | 94.20 | 0.90 | 91.94 | 1.35 | 2.26 | 1.02 | ** |
| 367-385 | 37.10 | 0.76 | 34.89 | 2.04 | 2.22 | 1.06 | * |
| 499-531 | 72.06 | 5.48 | 76.85 | 2.75 | −4.79 | 0.94 | * |
| 532-540 | 7.00 | 0.24 | 4.85 | 0.56 | 2.16 | 1.44 | *** |
| 559-569 | 5.17 | 1.00 | 3.93 | 0.77 | 1.24 | 1.31 | * |
| 570-577 | 28.67 | 0.69 | 26.69 | 2.20 | 1.98 | 1.07 | * |
| 578-600 | 20.13 | 0.54 | 17.24 | 2.84 | 2.89 | 1.17 | * |
| 584-600 | 51.23 | 0.73 | 46.30 | 3.49 | 4.93 | 1.11 | ** |
| 584-610 | 87.90 | 2.41 | 83.13 | 4.53 | 4.77 | 1.06 | * |
| 614-629 | 16.83 | 0.83 | 13.41 | 1.55 | 3.42 | 1.25 | *** |
| 669-689 | 99.84 | 0.01 | 99.88 | 0.01 | −0.03 | 1.00 | *** |
| 682-704 | 18.83 | 1.57 | 16.22 | 0.47 | 2.61 | 1.16 | ** |
| 690-694 | 0.47 | 0.11 | 0.37 | 0.09 | 0.11 | 1.29 | * |
| 694-704 | 59.11 | 2.39 | 63.48 | 1.79 | −4.37 | 0.93 | ** |
| 705-726 | 26.02 | 1.55 | 23.05 | 2.64 | 2.97 | 1.13 | * |
| 705-739 | 35.21 | 1.46 | 31.44 | 3.05 | 3.77 | 1.12 | ** |
| 727-739 | 12.04 | 1.75 | 9.20 | 1.58 | 2.85 | 1.31 | ** |
| 740-754 | 16.35 | 0.37 | 12.98 | 2.51 | 3.37 | 1.26 | ** |
| 740-753 | 44.06 | 1.69 | 38.00 | 3.84 | 6.07 | 1.16 | ** |
| 755-769 | 197.94 | 0.77 | 97.13 | 0.58 | 0.81 | 1.01 | * |

As shown in FIGS. 18 and 19 and Table 6 above, it was confirmed that the polypeptides consisting of the amino acids at positions 24 to 37, 38 to 45, 46 to 94, 141 to 154, 179 to 186, 216 to 226, 532 to 540, 559 to 569, 614 to 629, 690 to 694, 727 to 739, 740 to 754, respectively, in the extracellular domain of Lrig-1 protein represented by SEQ ID NO: 4, had an antigen-antibody conjugate/antigen ratio of 1.2 or more, suggesting that these polypeptides were likely to be selected as epitopes of the Lrig-1 protein. Furthermore, it was confirmed that the polypeptides consisting of the amino acid sequences represented by SEQ ID NOs: 14 to 22 showed a more significant actual difference value, and thus were more likely to be selected as epitopes.

### [8-1-3] Epitope Identification Using Asp-N+Lys-C

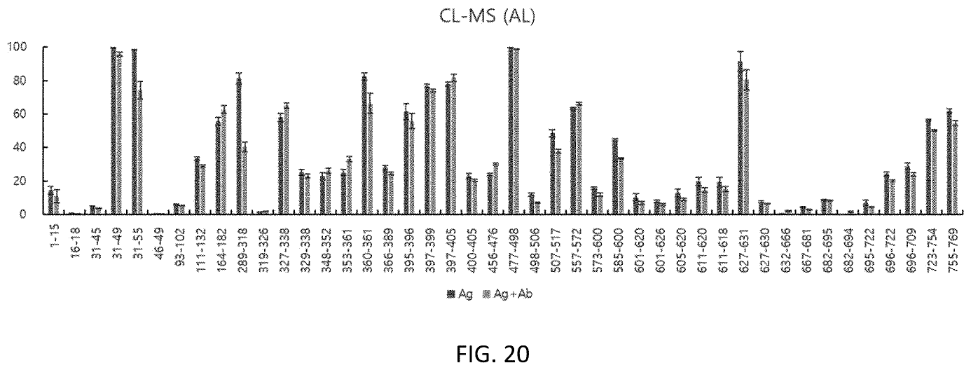

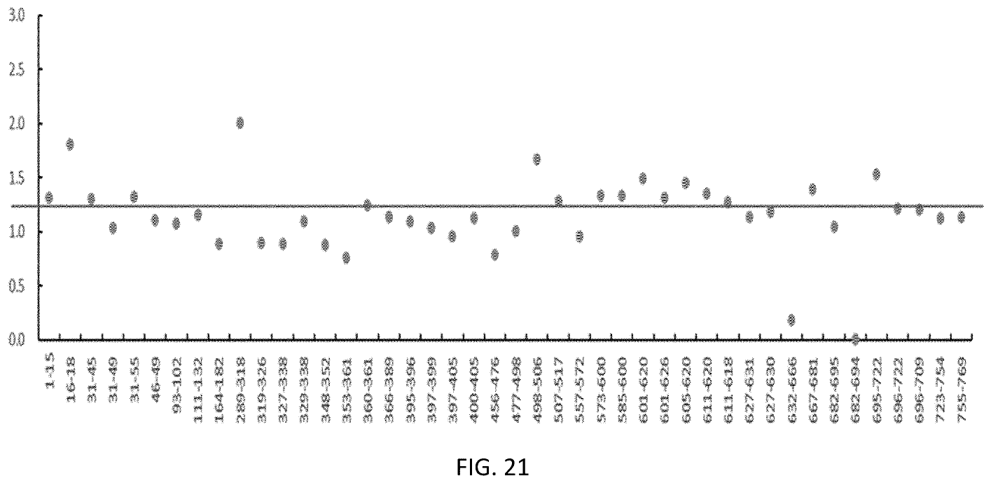

An experiment for epitope identification was conducted in the same manner as described in [8-1-1] above, except that Asp-N+Lys-C was used instead of chymotrypsin as the enzyme. The results are shown in FIGS. 20 and 21 and Table 7 below.

TABLE 7

| | | | Antigen- | | | | |
|---|---|---|---|---|---|---|---|
| Amino | | | antibody | | | | |
| acid | Antigen | | conjugate | | | | |
| positions | mean | S.D | mean | S.D | difference | Ratio | p value |
| −1-15 | 14.43 | 2.40 | 10.99 | 3.78 | 3.44 | 1.31 | * |
| 16-18 | 0.84 | 0.11 | 0.46 | 0.05 | 0.37 | 1.81 | ** |
| 31-45 | 5.01 | 0.20 | 3.83 | 0.08 | 1.18 | 1.31 | *** |
| 31-49 | 99.66 | 0.12 | 95.79 | 1.18 | 3.87 | 1.04 | *** |
| 31-55 | 98.23 | 0.17 | 74.26 | 5.16 | 23.98 | 1.32 | *** |
| 46-49 | 0.45 | 0.03 | 0.41 | 0.01 | 0.04 | 1.10 | ** |
| −93-102 | 5.74 | 0.41 | 5.35 | 0.12 | 0.38 | 1.07 | * |
| 111-132 | 33.35 | 0.92 | 28.93 | 0.61 | 4.42 | 1.15 | *** |
| 164-182 | 55.69 | 2.33 | 62.83 | 2.27 | −7.14 | 0.89 | *** |
| 289-318 | 81.31 | 3.28 | 40.46 | 2.89 | 40.86 | 2.01 | *** |
| 319-326 | 1.66 | 0.08 | 1.86 | 0.03 | −0.20 | 0.89 | *** |
| 327-338 | 57.92 | 2.43 | 65.19 | 1.55 | −7.27 | 0.89 | *** |
| 329-338 | 25.18 | 1.80 | 22.95 | 1.06 | 2.22 | 1.10 | ** |
| 348-352 | 22.88 | 2.16 | 26.19 | 1.63 | −3.32 | 0.87 | ** |
| 353-361 | 25.13 | 1.63 | 33.14 | 1.62 | −8.00 | 0.76 | *** |
| 360-361 | 82.60 | 2.14 | 66.35 | 6.14 | 16.24 | 1.24 | *** |
| 366-389 | 27.95 | 1.36 | 24.58 | 0.83 | 3.36 | 1.14 | *** |
| 395-396 | 61.42 | 4.84 | 55.89 | 4.32 | 15.52 | 1.10 | * |
| 397-399 | 76.77 | 0.99 | 73.96 | 0.83 | 2.82 | 1.04 | *** |
| 397-405 | 77.87 | 1.09 | 81.73 | 2.21 | −3.87 | 0.95 | ** |
| 400-405 | 22.98 | 1.52 | 20.48 | 0.55 | 2.50 | 1.12 | ** |
| 456-476 | 23.65 | 0.77 | 30.16 | 0.50 | −6.51 | 0.78 | *** |
| 477-498 | 100.00 | 0.00 | 98.88 | 0.04 | 1.12 | 1.01 | *** |
| 498-506 | 11.99 | 0.91 | 7.16 | 0.46 | 4.82 | 1.67 | *** |
| 507-517 | 48.71 | 2.15 | 37.83 | 1.31 | 10.87 | 1.29 | *** |
| 557-572 | 63.44 | 0.59 | 66.15 | 0.74 | −2.71 | 0.96 | *** |
| 573-600 | 15.68 | 0.87 | 11.78 | 0.98 | 3.90 | 1.33 | *** |
| 585-600 | 44.58 | 0.66 | 33.48 | 0.52 | 11.10 | 1.33 | *** |
| 601-620 | 10.27 | 2.06 | 6.88 | 0.83 | 3.39 | 1.49 | ** |
| 601-626 | 7.82 | 0.71 | 5.97 | 0.52 | 1.85 | 1.31 | *** |
| 605-620 | 12.99 | 2.31 | 8.95 | 0.88 | 4.03 | 1.45 | ** |
| 611-620 | 19.72 | 2.71 | 14.61 | 1.30 | 5.11 | 1.35 | *** |
| 611-618 | 19.38 | 2.98 | 15.17 | 1.53 | 4.22 | 1.28 | ** |
| 627-631 | 91.56 | 5.88 | 80.51 | 6.05 | 11.05 | 1.14 | ** |
| 627-630 | 7.59 | 0.53 | 6.42 | 0.20 | 1.17 | 1.18 | *** |
| 632-666 | 0.34 | 0.15 | 1.88 | 0.32 | −1.54 | 0.18 | *** |
| 667-681 | 4.25 | 0.29 | 3.04 | 0.11 | 1.21 | 1.40 | *** |
| 682-695 | 8.62 | 0.34 | 8.28 | 0.15 | 0.34 | 1.04 | * |
| 682-694 | 0.01 | 0.01 | 1.52 | 0.33 | −1.51 | 0.01 | *** |
| 695-722 | 7.06 | 1.46 | 4.61 | 0.28 | 2.45 | 1.53 | ** |
| 696-722 | 24.25 | 1.22 | 19.92 | 0.59 | 4.33 | 1.22 | *** |
| 696-709 | 28.84 | 2.10 | 23.96 | 0.97 | 4.88 | 1.20 | *** |
| 723-754 | 56.36 | 0.47 | 50.13 | 0.43 | 6.23 | 1.12 | *** |
| 755-769 | 61.79 | 1.18 | 54.57 | 1.37 | 7.22 | 1.13 | *** |

As shown in FIGS. 20 and 21 and Table 7 above, it was confirmed that the polypeptides consisting of the amino acids at positions 1 to 15, 16 to 18, 31 to 45, 31 to 55, 289 to 318, 360 to 361, 498 to 506, 507 to 517, 573 to 600, 585 to 600, 601 to 620, 601 to 626, 605 to 620, 611 to 618, 611 to 620, 667 to 681, 695 to 722, 696 to 709, and 696 to 722, respectively, in the extracellular domain of Lrig-1 protein represented by SEQ ID NO: 4, had an antigen-antibody conjugate/antigen ratio of 1.2 or more, suggesting that these polypeptides were likely to be selected as epitopes of the Lrig-1 protein. Furthermore, it was confirmed that the polypeptides consisting of the amino acid sequences represented by SEQ ID NOs: 23 to 28 showed a more significant actual difference value, and thus were more likely to be selected as epitopes.

As a result of performing additional analysis in a manner that overlaps the results of the epitope identification performed in [8-1-1] to [8-1-3] above, it could be seen that the polypeptides listed in the results were likely to be epitopes, and in particular, the polypeptides consisting of the amino acid sequences represented by SEQ ID NOs: 29 to 32 were more likely to be epitopes of the Lrig-1 protein for the GTC110-04 antibody.

### [8-2] Cross-Linking Mass Spectroscopy (XL-MS)

### [8-2-1] Epitope Identification Using Chymotrypsin

Two antigen-antibody conjugate samples were prepared by mixing the GTC110-04 antibody of Production Example 8 and the extracellular domain (antigen) of Lrig-1 protein represented by SEQ ID NO: 4 at a ratio of 2:1 and allowing the mixture to react sufficiently. Next, DSG (disuccinimidyl glutarate, 20593, Thermo) sufficiently dissolved in DMSO (dimethyl sulfoxide) was added to one of the samples at a ratio of 1:50 (sample: DSG) (T %), and only DMSO was added to the other sample (C %). Thereafter, each of the samples was allowed to react sufficiently at 25° C. for 1 hour. Then, in the same manner as in [8-1-1], each sample was treated with chymotrypsin and subjected to LC-MS analysis to obtain mass spectrometry values.

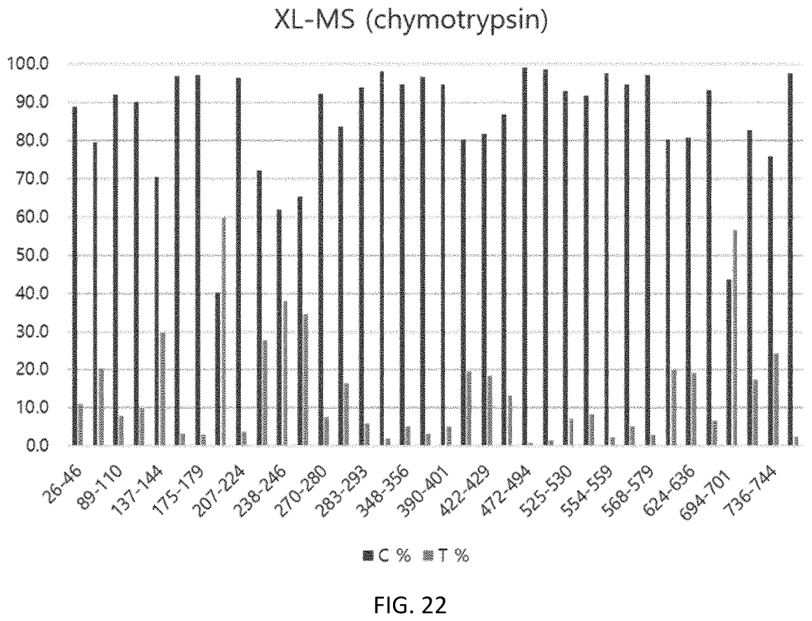

Thereafter, the difference between mass spectrometry values between the sample (T %) to which DSG was added and the sample (C %) to which only DMSO was added was examined. Among the epitopes having the difference, binding auxiliary regions (mono type and loop type) that were not epitopes were excluded, and regions having a difference of 10% or less were finally selected. The results are shown in FIG. 22 and Table 8 below.

TABLE 8

| Amino acid | | | | | |
|---|---|---|---|---|---|
| positions | C % | T % | Linker site | C % | T % + M/L |
| 26-46 | 88.9 | 11.1 | T36 | 50.8 | 49.2 |
| 66-86 | 79.6 | 20.4 | | | |
| 89-110 | 92.0 | 8.0 | K102 | 69.2 | 30.8 |
| 126-136 | 90.1 | 9.9 | | | |
| 137-144 | 70.5 | 29.5 | | | |
| 158-174 | 96.8 | 3.2 | K163 | 65.1 | 34.9 |
| 175-179 | 97.1 | 2.9 | K175 | 52.5 | 47.5 |
| 198-208 | 40.2 | 59.8 | | | |
| 207-224 | 96.4 | 3.6 | K207 | 82.3 | 17.7 |
| 222-237 | 72.2 | 27.8 | | | |
| 238-246 | 62.0 | 38.0 | | | |
| 252-269 | 65.4 | 34.6 | K267 | 47.2 | 52.8 |
| 270-280 | 92.2 | 7.8 | K274 | 87.3 | 12.7 |
| 279-282 | 83.6 | 16.4 | | | |
| 283-293 | 94.0 | 6.0 | T286 | 50.2 | 49.8 |
| 305-344 | 98.0 | 2.0 | K318 | 55.2 | 44.8 |
| 348-356 | 94.7 | 5.3 | K352 | 64.5 | 35.5 |
| 357-368 | 96.7 | 3.3 | K359, 365 | 88.7 | 11.3 |
| 390-401 | 94.7 | 5.3 | K394 | 85.7 | 14.3 |
| 417-420 | 80.3 | 19.7 | | | |
| 422-429 | 81.7 | 18.3 | | | |
| 441-453 | 86.7 | 13.3 | K441 | 74.7 | 25.3 |
| 472-494 | 99.1 | 10.9 | K476 | 97.1 | 2.9 |
| 495-511 | 98.5 | 1.5 | K497 | 98.2 | 1.8 |
| 525-530 | 92.9 | 7.1 | T525 | 92.7 | 7.3 |
| 531-541 | 91.7 | 18.3 | Y541 | 78.9 | 21.1 |
| 554-559 | 97.7 | 2.3 | K556 | 83.1 | 16.9 |
| 560-567 | 94.7 | 5.3 | T560 | 92.7 | 7.3 |
| 568-579 | 97.1 | 2.9 | T568, K569 | 94.9 | 5.1 |
| 585-598 | 80.2 | 19.8 | | | |
| 624-636 | 80.8 | 19.2 | T626 | 80.7 | 19.3 |
| 680-693 | 93.3 | 6.7 | | 93.3 | 6.7 |
| 694-701 | 43.5 | 56.5 | | | |
| 715-727 | 82.6 | 17.4 | | | |
| 736-744 | 75.8 | 24.2 | | | |
| 745-762 | 97.5 | 2.5 | K754 | 91.8 | 8.2 |

As shown in FIG. 22 and Table 8 above, it was confirmed that the polypeptides consisting of the amino acids at positions 472 to 494 (SEQ ID NO: 32), 495 to 511 (SEQ ID NO: 33), 525 to 530 (SEQ ID NO: 34), 560 to 567 (SEQ ID NO: 35), 568 to 579 (SEQ ID NO: 36), 680 to 693 (SEQ ID NO: 37), and 745 to 762 (SEQ ID NO: 38), respectively, in the extracellular domain of Lrig-1 protein represented by SEQ ID NO: 4, showed a very low crosslinking level of 5% or less or 10% or less, suggesting that these polypeptides were likely to be selected as epitopes of the Lrig-1 protein.

### [8-2-2] Epitope Identification Using Trypsin

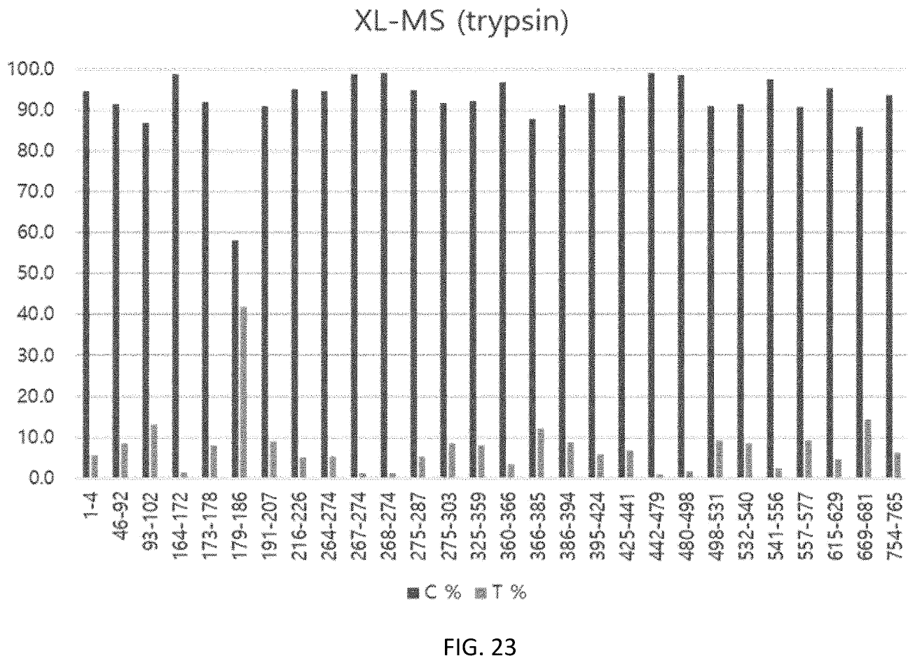

An experiment for epitope identification was conducted in the same manner as described in [8-2-1] above, except that trypsin was used instead of chymotrypsin as the enzyme. The results are shown in FIG. 23 and Table 9 below.

TABLE 9

| Amino acid | | | | | |
|---|---|---|---|---|---|
| positions | C % | T % | Linker site | C % | T % + M/L |
| 1-4 | 94.6 | 5.4 | | 94.6 | 5.4 |
| 46-92 | 91.4 | 8.6 | | 91.4 | 8.6 |
| 93-102 | 86.8 | 13.2 | | | |
| 164-172 | 98.6 | 1.4 | | 98.6 | 1.4 |
| 173-178 | 91.9 | 8.1 | K175 | 73.8 | 26.2 |
| 179-186 | 58.1 | 41.9 | | | |
| 191-207 | 91.0 | 9.0 | K207 | 77.0 | 23.0 |
| 216-226 | 95.0 | 5.0 | K215 | 88.6 | 11.4 |
| 264-274 | 94.6 | 5.4 | | 94.6 | 5.4 |
| 267-274 | 98.7 | 1.3 | K267 | 92.9 | 7.1 |
| 268-274 | 98.9 | 1.1 | | 98.9 | 1.1 |
| 275-287 | 94.8 | 5.2 | | 94.8 | 5.2 |
| 275-303 | 91.6 | 8.4 | | 91.6 | 8.4 |
| 325-359 | 92.1 | 7.9 | K352 | 89.9 | 10.1 |
| 360-366 | 96.7 | 3.3 | K361, 365 | 58.0 | 42.0 |
| 366-385 | 87.8 | 12.2 | | | |
| 386-394 | 91.3 | 8.7 | | 91.3 | 8.7 |
| 395-424 | 94.2 | 5.8 | K396, 415 | 93.6 | 6.4 |
| 425-441 | 93.3 | 6.7 | | 93.3 | 6.7 |
| 442-479 | 99.0 | 1.0 | | 99.0 | 1.0 |
| 480-498 | 98.4 | 1.6 | | 98.4 | 1.6 |
| 498-531 | 90.9 | 9.1 | | 90.9 | 9.1 |
| 532-540 | 91.4 | 8.6 | | 91.4 | 8.6 |
| 541-556 | 97.5 | 2.5 | K556 | 96.3 | 3.7 |
| 557-577 | 90.8 | 9.2 | K569 | 87.9 | 12.1 |
| 615-629 | 95.3 | 4.7 | | 95.3 | 4.7 |
| 669-681 | 85.8 | 14.2 | | | |
| 754-765 | 93.6 | 6.4 | K754 | 53.0 | 47.0 |

As shown in FIG. 23 and Table 9 above, it was confirmed that the polypeptides consisting of the amino acids at positions 1 to 4 (SEQ ID NO: 80), 46 to 92 (SEQ ID NO: 40), 164 to 172 (SEQ ID NO: 41), 264 to 274 (SEQ ID NO: 42), 267 to 274 (SEQ ID NO: 43), 268 to 274 (SEQ ID NO: 44), 275 to 287 (SEQ ID NO: 45), 275 to 303 (SEQ ID NO: 46), 386 to 394 (SEQ ID NO: 47), 395 to 424 (SEQ ID NO: 48), 425 to 441 (SEQ ID NO: 49), 442 to 479 (SEQ ID NO: 50), 480 to 498 (SEQ ID NO: 51), 498 to 531 (SEQ ID NO: 52), 532 to 540 (SEQ ID NO: 53), 541 to 556 (SEQ ID NO: 54), and 615 to 629 (SEQ ID NO: 55), respectively, in the extracellular domain of Lrig-1 protein represented by SEQ ID NO: 4, showed a very low crosslinking level of 5% or less or 10% or less, suggesting that these polypeptides were likely to be selected as epitopes of the Lrig-1 protein.

### [8-2-3] Epitope Identification Using Asp-N+Lys-C

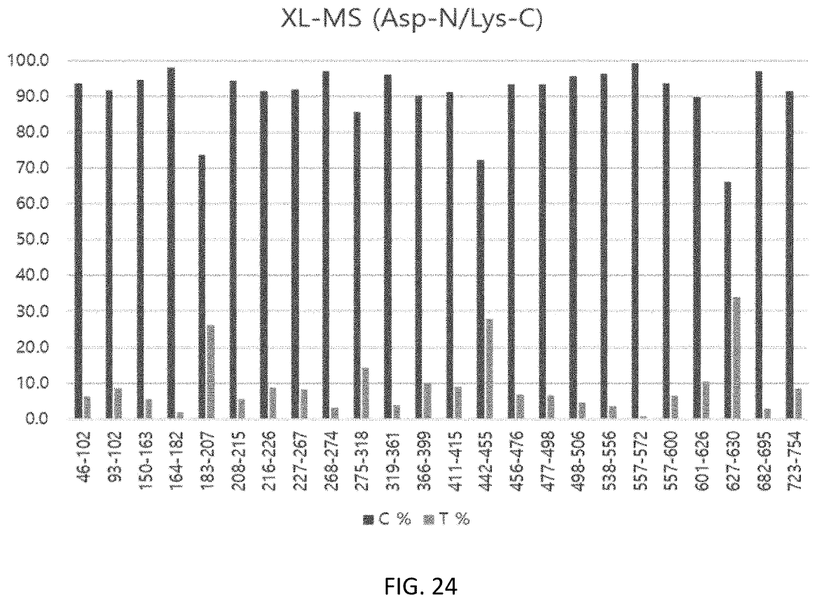

An experiment for epitope identification was conducted in the same manner as described in [8-2-1] above, except that Asp-N+Lys-C was used instead of chymotrypsin as the enzyme. The results are shown in FIG. 24 and Table 10 below.

TABLE 10

| Amino acid | | | | | |
|---|---|---|---|---|---|
| positions | C % | T % | Linker site | C % | T % + M/L |
| 46-102 | 93.7 | 6.3 | | 93.7 | 6.3 |
| 93-102 | 91.6 | 8.4 | | 91.6 | 8.4 |
| 150-163 | 94.5 | 5.5 | | 94.5 | 5.5 |
| 164-182 | 98.0 | 2.0 | | 98.0 | 2.0 |
| 183-207 | 73.7 | 26.3 | | | |
| 208-215 | 94.3 | 5.7 | K215 | 85.1 | 14.9 |
| 216-226 | 91.4 | 8.6 | | 91.4 | 8.6 |
| 227-267 | 91.8 | 8.2 | | 91.8 | 8.2 |
| 268-274 | 97.0 | 3.0 | | 97.0 | 3.0 |
| 275-318 | 85.6 | 14.4 | | | |
| 319-361 | 96.0 | 4.0 | K352, 361 | 91.6 | 8.4 |
| 366-399 | 90.1 | 9.9 | K394, 396 | 89.9 | 10.1 |
| 411-415 | 91.1 | 8.9 | | 91.1 | 8.9 |
| 442-455 | 72.2 | 27.8 | | | |
| 456-476 | 93.3 | 6.7 | | 93.3 | 6.7 |
| 477-498 | 93.4 | 6.6 | | 93.4 | 6.6 |
| 498-506 | 95.5 | 4.5 | | 95.5 | 4.5 |
| 538-556 | 96.4 | 3.6 | | 96.4 | 3.6 |
| 557-572 | 99.3 | 0.7 | T560 | 98.5 | 1.5 |
| 557-600 | 93.6 | 6.4 | | 93.6 | 6.4 |
| 601-626 | 89.7 | 10.3 | | | |
| 627-630 | 66.0 | 34.0 | K629 | 64.7 | 35.3 |
| 682-695 | 97.1 | 2.9 | K694 | 79.4 | 20.6 |
| 723-754 | 91.4 | 8.6 | K754 | 84.1 | 15.9 |

As shown in FIG. 24 and Table 10 above, it was confirmed that the polypeptides consisting of the amino acids at positions 46 to 102 (SEQ ID NO: 56), 93 to 102 (SEQ ID NO: 57), 150 to 163 (SEQ ID NO: 58), 164 to 182 (SEQ ID NO: 59), 216 to 226 (SEQ ID NO: 60), 227 to 267 (SEQ ID NO: 61), 268 to 274 (SEQ ID NO: 62), 319 to 361 (SEQ ID NO: 63), 411 to 415 (SEQ ID NO: 64), 456 to 476 (SEQ ID NO: 65), 477 to 498 (SEQ ID NO: 66), 498 to 506 (SEQ ID NO: 67), 538 to 556 (SEQ ID NO: 68), 557 to 572 (SEQ ID NO: 69), and 557 to 600 (SEQ ID NO: 70), respectively, in the extracellular domain of Lrig-1 protein represented by SEQ ID NO: 4, showed a very low crosslinking level of 5% or less or 10% or less, suggesting that these polypeptides were likely to be selected as epitopes of the Lrig-1 protein.

As a result of performing additional analysis in a manner that overlaps the results of the epitope identification performed in [8-2-1] to [8-2-3] above, it could be seen that the polypeptides listed in the results were likely to be epitopes, and in particular, the polypeptide consisting of the amino acid sequences represented by any one of SEQ ID NOs: 32 to 36, SEQ ID NOs: 40 to 42, and SEQ ID NOs: 64 and 68 was more likely to be an epitope of the Lrig-1 protein for the GTC110-04 antibody.

### [8-3] Epitope Prediction

Based on the sequence of specific epitope sequences obtained through the process of overlapping the results of epitope identification obtained in [8-1] and [8-2] above, an experiment for epitope identification was conducted in the same manner as in [8-1] above. The results are shown in Tables 11 and 12 below.

TABLE 11

| | | | Antigen- | | |
|---|---|---|---|---|---|
| | Amino acid | | antibody | | |
| Enzyme | positions | Antigen | conjugate | Difference | Ratio |
| Asp-N Lys-C | 31-45 | 5.01 | 3.83 | 1.18 | 1.31 |
| Asp-N Lys-C | 31-55 | 98.23 | 74.26 | 23.98 | 1.32 |
| Chymotrypsin | 36-64 | 51.37 | 40.77 | 10.60 | 1.26 |
| Trypsin | 38-45 | 5.70 | 4.15 | 1.55 | 1.37 |
| Trypsin | 46-94 | 91.22 | 74.75 | 16.47 | 1.22 |

TABLE 12

| | | | Antigen- | | |
|---|---|---|---|---|---|
| | Amino acid | | antibody | | |
| Enzyme | positions | Antigen | conjugate | Difference | Ratio |
| Trypsin | 24-37 | 1.67 | 1.11 | 0.56 | 1.51 |
| Chymotrypsin | 26-64 | 59.56 | 51.51 | 8.05 | 1.16 |
| Asp-N Lys-C | 31-49 | 99.66 | 95.79 | 3.87 | 1.04 |
| Chymotrypsin | 38-64 | 53.47 | 46.30 | 7.16 | 1.15 |
| Asp-N Lys-C | 62-92 | 65.30 | 65.51 | −0.22 | 1.00 |
| Chymotrypsin | 65-87 | 47.90 | 43.09 | 4.81 | 1.11 |
| Chymotrypsin | 88-94 | 89.93 | 89.48 | 0.44 | 1.00 |

As shown in Tables 11 and 12, it was confirmed that the polypeptide consisting of the amino acids at positions 46 to 87 or 46 to 64 in the extracellular domain of Lrig-1 protein represented by SEQ ID NO: 4 showed a great difference in the ratio in the results obtained using various enzymes.

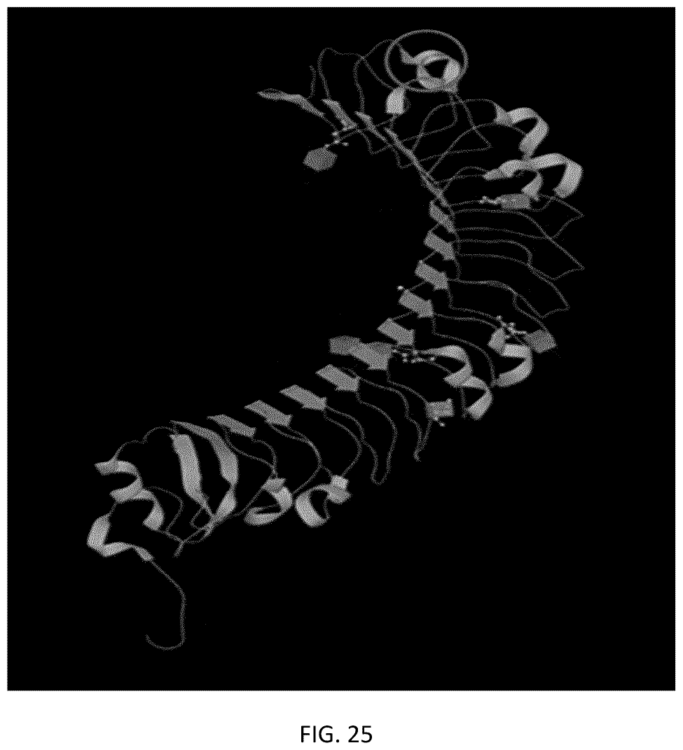

From the above results, it can be seen that the GTC110-04 antibody may bind specifically to the polypeptide consisting of the amino acids at positions 46 to 87 or 46 to 64 in the amino acid sequence of the extracellular domain of the Lrig-1 protein (circled region in FIG. 25).

Although the present invention has been described in detail above, the scope of the present invention is not limited thereto, and it will be apparent to those of ordinary skill in the art that various modifications and changes are possible without departing from the technical spirit of the present invention as defined in the appended claims.

### INDUSTRIAL APPLICABILITY

The antibody or antigen-binding fragment that binds specifically to the epitope of Lrig-1 protein according to the present invention may bind specifically to the epitope of the present invention, which is present on regulatory T cells, thereby inhibiting the function of the regulatory T cells and maintaining or increasing the activity of effector T cells. Thus, it may be very efficiently used for the prevention, amelioration or treatment of cancer.

1. An epitope that is an epitope of Lrig-1 (leucine-rich and immunoglobulin-like domains 1) protein comprising at least one polypeptide selected from the group consisting of polypeptides consisting of the amino acid sequences represented by SEQ ID NOs: 7 to 70; or an epitope of Lrig-1 (leucine-rich and immunoglobulin-like domains 1) protein comprising a polypeptide consisting of the amino acid sequences represented by SEQ ID NO: 71 or SEQ ID NO: 72.

2. (canceled)

3. An antibody or antigen-binding fragment that binds specifically to an epitope comprising at least one polypeptide selected from the group consisting of polypeptides consisting of the amino acid sequences represented by SEQ ID NOS: 7 to 70.

4. An antibody or antigen-binding fragment that binds specifically to an epitope comprising a polypeptide consisting of the amino acid sequence represented by SEQ ID NO: 71 or SEQ ID NO: 72.

5. A nucleic acid molecule encoding the epitope of claim 1.

6. An expression vector into which the nucleic acid molecule of claim 5 has been inserted.

7. A host cell line transfected with the expression vector of claim 6.

8. A pharmaceutical composition for preventing or treating cancer comprising the antibody or antigen-binding fragment of claim 3 as an active ingredient.

9. The pharmaceutical composition of claim 8, wherein the pharmaceutical composition is for preventing or treating cancer and wherein the cancer is gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, or lung cancer.

10. An antibody-drug conjugate comprising: the antibody or antigen-binding fragment of claim 3; and a drug.

11. A method for preventing or treating cancer comprising a step of administering, to a subject, a pharmaceutically effective amount of an antibody or antigen-binding fragment of claim 3.

12. A method for preventing or treating cancer comprising a step of administering, to a subject, a pharmaceutically effective amount of an antibody or antigen-binding fragment of claim 4.

13. The antibody or antigen-binding fragment thereof of claim 3, wherein the antibody or antigen-binding fragment comprises:
an antibody heavy chain variable region (VH) comprising:
a VHCDR1 having the amino acid sequence set forth in SEQ ID NO: 107 or the amino acid sequence set forth in SEQ ID NO: 113 or amino acid sequence set forth in SEQ ID NO: 119 or amino acid sequence set forth in SEQ ID NO: 125;
a VHCDR2 having the amino acid sequence set forth in SEQ ID NO: 108 or the amino acid sequence set forth in SEQ ID NO: 114 or amino acid sequence set forth in SEQ ID NO: 120 or amino acid sequence set forth in SEQ ID NO: 126; and
a VHCDR3 having the amino acid sequence set forth in SEQ ID NO: 109 or the amino acid sequence set forth in SEQ ID NO: 115 or amino acid sequence set forth in SEQ ID NO: 121 or amino acid sequence set forth in SEQ ID NO: 127; and
an antibody light chain variable region (VL) comprising:
a VLCDR1 having the amino acid sequence set forth in SEQ ID NO: 110 or the amino acid sequence set forth in SEQ ID NO: 116 or amino acid sequence set forth in SEQ ID NO: 122 or amino acid sequence set forth in SEQ ID NO: 128;
a VLCDR2 having the amino acid sequence set forth in SEQ ID NO: 111 or the amino acid sequence set forth in SEQ ID NO: 117 or amino acid sequence set forth in SEQ ID NO: 123 or amino acid sequence set forth in SEQ ID NO: 129; and
a VLCDR3 having the amino acid sequence set forth in SEQ ID NO: 112 or the amino acid sequence set forth in SEQ ID NO: 118 or amino acid sequence set forth in SEQ ID NO: 124 or amino acid sequence set forth in SEQ ID NO: 130.

14. The antibody or antigen-binding fragment thereof of claim 3, wherein the antibody or antigen-binding fragment binds to Lrig-1, wherein said antibody or antigen-binding fragment thereof binds to Lrig-1 by binding at least to an epitope within Lrig-1 having at least one polypeptide selected from the group consisting of polypeptides consisting of the amino acid sequences represented by SEQ ID NOs: 7 to 70.

15. The antibody or antigen-binding fragment thereof of claim 4, wherein the antibody or antigen-binding fragment binds to Lrig-1, wherein said antibody or antigen-binding fragment thereof binds to Lrig-1 by binding at least to an epitope within Lrig-1 having the amino acid represented by SEQ ID NO: 71 or SEQ ID NO: 72.

16. A method of screening an antibody or antigen-binding fragment by using the determined epitopes of Lrig-1, wherein the determined epitopes of Lrig-1 comprise at least one epitope of claim 1.

17. A method of binding an antibody or antigen-binding fragment to determined epitopes of Lrig-1, wherein the determined epitopes of Lrig-1 comprise at least one epitope of claim 1.

18. A pharmaceutical composition for preventing or treating cancer comprising the antibody or antigen-binding fragment of claim 4 as an active ingredient.

19. The pharmaceutical composition of claim 18, wherein the pharmaceutical composition is for preventing or treating cancer and wherein the cancer is gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, or lung cancer.

20. An antibody-drug conjugate comprising: the antibody or antigen-binding fragment of claim 4; and a drug.

21. The antibody or antigen-binding fragment thereof of claim 4, wherein the antibody or antigen-binding fragment comprises:
an antibody heavy chain variable region (VH) comprising:
a VHCDR1 having the amino acid sequence set forth in SEQ ID NO: 107 or the amino acid sequence set forth in SEQ ID NO: 113 or amino acid sequence set forth in SEQ ID NO: 119 or amino acid sequence set forth in SEQ ID NO: 125;
a VHCDR2 having the amino acid sequence set forth in SEQ ID NO: 108 or the amino acid sequence set forth in SEQ ID NO: 114 or amino acid sequence set forth in SEQ ID NO: 120 or amino acid sequence set forth in SEQ ID NO: 126; and
a VHCDR3 having the amino acid sequence set forth in SEQ ID NO: 109 or the amino acid sequence set forth in SEQ ID NO: 115 or amino acid sequence set forth in SEQ ID NO: 121 or amino acid sequence set forth in SEQ ID NO: 127; and
an antibody light chain variable region (VL) comprising:
a VLCDR1 having the amino acid sequence set forth in SEQ ID NO: 110 or the amino acid sequence set forth in SEQ ID NO: 116 or amino acid sequence set forth in SEQ ID NO: 122 or amino acid sequence set forth in SEQ ID NO: 128;
a VLCDR2 having the amino acid sequence set forth in SEQ ID NO: 111 or the amino acid sequence set forth in SEQ ID NO: 117 or amino acid sequence set forth in SEQ ID NO: 123 or amino acid sequence set forth in SEQ ID NO: 129; and
a VLCDR3 having the amino acid sequence set forth in SEQ ID NO: 112 or the amino acid sequence set forth in SEQ ID NO: 118 or amino acid sequence set forth in SEQ ID NO: 124 or amino acid sequence set forth in SEQ ID NO: 130.


SEQUENCE LISTING (164 sequences)

SEQ ID NO: 1 - protein, 1093 aa, Homo sapiens [sequence omitted]

SEQ ID NO: 2 - DNA, 3282 bp, Homo sapiens
atggcgcggccggtccggggagggctcggggccccgcgccgctcgccttgccttctccttctctggctgcttttgcttcggctggagccggtgaccgccgcggccggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccatggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacttgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtcgtgccaccagatgttccaagctacctctcttctcaggggaccctttctgaccgacaagaaaccgtggtcaggaccgagggtggccctcaggccaatgggcacattgagagcaatggtgtgtgtccaagagatgcaagccactttccagagcccgacactcacagcgttgcctgcaggcagccaaagctctgtgctgggtctgcgtatcacaaagagccgtggaaagcgatggagaaagctgaagggacacctgggccacataagatggaacacggtggccgggtcgtatgcagtgactgcaacaccgaagtggactgttactccaggggacaagccttccacccccagcctgtgtccagagacagcgcacagccaagtgcgccaaatggcccggagccgggtgggagtgaccaagagcattctccacatcaccagtgcagcaggactgccgctgggtcctgccccgagtgccaagggtcgctctaccccagtaaccacgatagaatgctgacggctgtgaagaaaaagccaatggcatctctagatgggaaaggggattcttcctggactttagcaaggttgtatcacccggactccacagagctacagcctgcatcttcattaacttcaggcagtccagagcgcgcggaagcccagtacttgcttgtttccaatggccacctccccaaagcatgtgacgccagtcccgagtccacgccactgacaggacagctccccgggaaacagagggtgccactgctgttggcaccaaaaagctag

SEQ ID NO: 3 - protein, 1091 aa, Mus musculus
MARPGPGVLGAPRLAPRLLLWLLLLLLQWPESAGAQAGPRAPCAAACTCAGDSLDCSGRGLATLPRDLPSWTRSLNLSYNRLSEIDSAAFEDLTNLQEVYLNSNELTAIPSLGTASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTVGIFTIAVVCSIVLTSLVWVCIIYQTRKKSEEYSVTNTDETIVPPDVPSYLSSQGTLSDRQETVVRTEGGHQANGHIESNGVCLRDPSLFPEVDIHSTTCRQPKLCVGYTREPWKVTEKADRTAAPHTTAHSGSAVCSDCSTDTAYHPQPVPRDSGQPGTASSQELRQHDREYSPHHPYSGTADGSHTLSGGSLYPSNHDRILPSLKNKAASADGNGDSSWTLAKLHEADCIDLKPSPTLASGSPELMEDAISTEAQHLLVSNGHLPKACDSSPESVPLKGQITGKRRGPLLLAPRS

SEQ ID NO: 4 - DNA, 3276 bp, Mus musculus
atggcgcggcccggtccgggagtgctcggagccccgcgccttgcgcctcgccttctgctctggctgctcttgctgctactgcaatggccggagtcagcgggcgcccaggctcgcccgcgggccccctgcgcggccgcctgcacttgcgccgggaactcgctggactgcagtgggcgcgggctggcgacgctgccccgggacctgccctcctggacgcgcagcctaaacctgagttataacagactctccgagatcgactctgctgcttttgaggacttgacgaatctgcaggaagtgtacctcaacagcaatgagctgacagccataccatcactgggcactgcttccataggagttgtctctctctttttgcagcacaacaagatccttagtgtggatgggagccagctgaagtcgtacctgtccttggaagtgctggatctgagttccaacaacatcacggaaattcggagctcctgtttcccgaacggcctgcgtataagggaactcaacttggcgagcaaccgcatcagcatcctggagtctggagcatttgatggtctgtcgcggtcactgctgactctccgtctgagcaaaaacaggatcacccagcttcctgtgaaagcgttcaagctacccaggctgacacaactagacctgaatcggaatcggattcggctgattgaaggcctcacgttccaggggctcgacagcttagaggtgctgaggcttcagaggaacaacatcagcaggctgacggacggggccttctgggggctgtctaagatgcacgtgctgcacctggagtacaacagtctggtggaagtgaacagtggctccctctatggcctcacagccctgcaccagctgcacctcagcaacaactccatctctcgaattcagcgtgatggctggagcttctgccaaaagctgcatgagttgattctgtccttcaacaacctcacgcggctggatgaggagagtctagcggagttgagcagcctcagtatcctgcgcctcagtcacaacgccatcagtcacattgctgaaggcgccttcaagggactcaagagtctgcgggtcttggacctggaccataacgagatctcgggtacaatcgaggataccagtggtgcctttacggggcttgacaacctcagcaagctgactctgtttggaaacaagatcaaatctgtggctaagagagccttctcgggcctggaaagcctggaacacctgaaccttggagagaatgcaatcaggtctgtccagtttgatgcctttgcaaagatgaagaaccttaaagagctctacatcagcagtgagagcttcctgtgtgactgccagctcaagtggctgcccccatggctaatgggtaggatgctgcaggcctttgtgacagccacctgtgcccatccagagtcgctgaagggccagagcattttctcagtgctgccagacagctttgtgtgtgatgactttccaaagccacagatcatcacccagcctgagacgaccatggctgtggtgggcaaggacatccgtttcacatgctccgcagccagcagcagcagctcaccaatgaccttcgcctggaagaaggacaatgaggtcctggccaatgcagacatggagaactttgcccacgtccgtgcacaggacggcgaagtgatggagtataccactatcctgcacctccgtcacgtcacctttgggcacgagggccgctaccagtgtatcatcacaaaccactttggctccacatactcccacaaagccaggctcactgtgaatgtgttgccatcattcactaaaataccccatgacattgccatccggactggcaccacagcccgcctcgagtgtgctgccacgggccaccctaaccctcagattgcctggcagaaggatggaggcaccgatttcccggcagctcgtgagcgacgcatgcatgttatgccagacgatgatgtgttcttcatcactgatgtgaaaatagacgacatgggggtctacagctgcactgcccagaactcggcaggctcggtttcagccaacgctaccctcacagtcttagaaactccatccttggcagtgcctctggaagaccgtgtggtaactgtgggagaaacagtggccttccagtgcaaagcaaccgggagccccacaccacgcatcacctggcttaagggaggtcgcccattgagcctcacagagcgccaccatttcactccaggcaaccagctgctggttgttcagaatgtgatgatagacgatgcagggcggtatacctgtgagatgtctaatcccctgggcactgagcgagcacatagccagctgagcattttacctacccctggctgccggaaggatgggaccaccgtaggcatcttcaccattgctgtggtgtgcagtattgtcctgacctcgctggtgtgggtgtgcatcatctaccagaccaggaagaagagcgaggagtacagcgtcactaacacagatgaaaccattgtgcctccagatgttccaagctacctctcctctcagggaaccctttctgaccggcaggaaactgtggtcaggactgagggtggtcatcaggccaatgggcacattgaaagcaatggtgtgtgtctgagagacccgagcctcttcccagaggtcgacattcatagcactacatgtaggcagcccaagctgtgtgttggatacactagagagccctggaaggtgacggagaaagccgacaggacagctgccccacatacaacagcacacagtggctctgctgtatgtagtgactgcagtactgacacagcctaccatccccagcctgtgcccagagacagtgggcagccaggcacagcgagcagccaagagctcaggcagcatgaccgggaatattctccacaccatccttacagtgggactgcggatgggtctcataccctctctggggggtccctctatccaagcaaccatgacagaatactgccatccttgaagaacaaggcggcgtctgcagatgggaacggagattcctcttggactttagcaaagttacatgaagcagactgcatagacctaaagccttctcctacgttagcttcaggcagcccagagctcatggaagacgccatatctactgaagcccagcacttgcttgtttccaatggccacctccccaaggcctgtgactccagccctgaatctgtgccactgaaagggcagatcactgggaaacggaggggaccactgctattggcaccgagaagctag

SEQ ID NO: 5 - protein, 762 aa, Homo sapiens
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIF

SEQ ID NO: 6 - protein, 760 aa, Mus musculus
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTT

SEQ ID NO: 7 - protein, 29 aa, Artificial Sequence
  Epitope
TRSLNLSYNKLSEIDPAGFEDLPNLQEVY

SEQ ID NO: 8 - protein, 31 aa, Artificial Sequence
  Epitope
YLSLEVLDLSLNNITEVRNTCFPHGPPIKEL

SEQ ID NO: 9 - protein, 3 aa, Artificial Sequence
  Epitope
HQL

SEQ ID NO: 10 - protein, 19 aa, Artificial Sequence
  Epitope
DHNEISGTIEDTSGAFSGL

SEQ ID NO: 11 - protein, 7 aa, Artificial Sequence
  Epitope
HLRQVTF

SEQ ID NO: 12 - protein, 38 aa, Artificial Sequence
  Epitope
QKDGGTDFPAARERRMHVMPDDDVFFITDVKIDDAGVY

SEQ ID NO: 13 - protein, 36 aa, Artificial Sequence
  Epitope
SLTERHHLTPDNQLLVVQNVVAEDAGRYTCEMSNTL

SEQ ID NO: 14 - protein, 71 aa, Artificial Sequence
  Epitope
GLAALPGDLPSWTRSLNLSYNKLSEIDPAGFEDLPNLQEVYLNNNELTAVPSLGAASSHVVSLFLQHNKIR

SEQ ID NO: 15 - protein, 14 aa, Artificial Sequence
  Epitope
IGTLELGAFDGLSR

SEQ ID NO: 16 - protein, 8 aa, Artificial Sequence
  Epitope
LTQLDLNR

SEQ ID NO: 17 - protein, 11 aa, Artificial Sequence
  Epitope
LTDGAFWGLSK

SEQ ID NO: 18 - protein, 9 aa, Artificial Sequence
  Epitope
QVTFGHEGR

SEQ ID NO: 19 - protein, 11 aa, Artificial Sequence
  Epitope
LTVNVLPSFTK

SEQ ID NO: 20 - protein, 16 aa, Artificial Sequence
  Epitope
MHVMPDDDVFFITDVK

SEQ ID NO: 21 - protein, 5 aa, Artificial Sequence
  Epitope
ITWFK

SEQ ID NO: 22 - protein, 28 aa, Artificial Sequence
  Epitope
YTCEMSNTLGTERAHSQLSVLPAAGCRK

SEQ ID NO: 23 - protein, 18 aa, Artificial Sequence
  Epitope
AGPRAPCAAACTCAGDSL

SEQ ID NO: 24 - protein, 25 aa, Artificial Sequence
  Epitope
DLPSWTRSLNLSYNKLSEIDPAGFE

SEQ ID NO: 25 - protein, 30 aa, Artificial Sequence
  Epitope
DEESLAELSSLSVLRLSHNSISHIAEGAFK

SEQ ID NO: 26 - protein, 20 aa, Artificial Sequence
  Epitope
KDNEVLTNADMENFVHVHAQ

SEQ ID NO: 27 - protein, 54 aa, Artificial Sequence
  Epitope
DITIRTTTVARLECAATGHPNPQIAWQKDGGTDFPAARERRMHVMPDDDVFFIT

SEQ ID NO: 28 - protein, 28 aa, Artificial Sequence
  Epitope
GDRPLSLTERHHLTPDNQLLVVQNVVAE

SEQ ID NO: 29 - protein, 29 aa, Artificial Sequence
  Epitope
TRSLNLSYNKLSEIDPAGFEDLPNLQEVY

SEQ ID NO: 30 - protein, 2 aa, Artificial Sequence
  Epitope
QK

SEQ ID NO: 31 - protein, 14 aa, Artificial Sequence
  Epitope
ERRMHVMPDDDVFF

SEQ ID NO: 32 - protein, 23 aa, Artificial Sequence
  Epitope
AMVGKDIRFTCSAASSSSSPMTF

SEQ ID NO: 33 - protein, 17 aa, Artificial Sequence
  Epitope
AWKKDNEVLTNADMENF

SEQ ID NO: 34 - protein, 6 aa, Artificial Sequence
  Epitope
TTILHL

SEQ ID NO: 35 - protein, 8 aa, Artificial Sequence
  Epitope
TVNVLPSF

SEQ ID NO: 36 - protein, 12 aa, Artificial Sequence
  Epitope
TKTPHDITIRTT

SEQ ID NO: 37 - protein, 14 aa, Artificial Sequence
  Epitope
CKATGNPPPRITWF

SEQ ID NO: 38 - protein, 18 aa, Artificial Sequence
  Epitope
SVLPAAGCRKDGTTVGIF

SEQ ID NO: 39 - protein, 4 aa, Artificial Sequence
  Epitope
AGPR

SEQ ID NO: 40 - protein, 47 aa, Artificial Sequence
  Epitope
LSEIDPAGFEDLPNLQEVYLNNNELTAVPSLGAASSHVVSLFLQHNK

SEQ ID NO: 41 - protein, 9 aa, Artificial Sequence
  Epitope
NRITQLPVR

SEQ ID NO: 42 - protein, 11 aa, Artificial Sequence
  Epitope
IHRKGWSFCQK

SEQ ID NO: 43 - protein, 8 aa, Artificial Sequence
  Epitope
KGWSFCQK

SEQ ID NO: 44 - protein, 7 aa, Artificial Sequence
  Epitope
GWSFCQK

SEQ ID NO: 45 - protein, 13 aa, Artificial Sequence
  Epitope
LHELVLSFNNLTR

SEQ ID NO: 46 - protein, 29 aa, Artificial Sequence
  Epitope
LHELVLSFNNLTRLDEESLAELSSLSVLR

SEQ ID NO: 47 - protein, 9 aa, Artificial Sequence
  Epitope
SVQFDAFVK

SEQ ID NO: 48 - protein, 30 aa, Artificial Sequence
  Epitope
MKNLKELHISSDSFLCDCQLKWLPPWLIGR

SEQ ID NO: 49 - protein, 17 aa, Artificial Sequence
  Epitope
MLQAFVTATCAHPESLK

SEQ ID NO: 50 - protein, 38 aa, Artificial Sequence
  Epitope
GQSIFSVPPESFVCDDFLKPQIITQPETTMAMVGKDIR

SEQ ID NO: 51 - protein, 19 aa, Artificial Sequence
  Epitope
FTCSAASSSSSPMTFAWKK

SEQ ID NO: 52 - protein, 34 aa, Artificial Sequence
  Epitope
KDNEVLTNADMENFVHVHAQDGEVMEYTTILHLR

SEQ ID NO: 53 - protein, 9 aa, Artificial Sequence
  Epitope
QVTFGHEGR

SEQ ID NO: 54 - protein, 16 aa, Artificial Sequence
  Epitope
YQCVITNHFGSTYSHK

SEQ ID NO: 55 - protein, 15 aa, Artificial Sequence
  Epitope
HVMPDDDVFFITDVK

SEQ ID NO: 56 - protein, 57 aa, Artificial Sequence
  Epitope
LSEIDPAGFEDLPNLQEVYLNNNELTAVPSLGAASSHVVSLFLQHNKIRSVEGSQLK

SEQ ID NO: 57 - protein, 10 aa, Artificial Sequence
  Epitope
IRSVEGSQLK

SEQ ID NO: 58 - protein, 14 aa, Artificial Sequence
  Epitope
DGLSRSLLTLRLSK

SEQ ID NO: 59 - protein, 19 aa, Artificial Sequence
  Epitope
NRITQLPVRAFKLPRLTQL

SEQ ID NO: 60 - protein, 11 aa, Artificial Sequence
  Epitope
LTDGAFWGLSK

SEQ ID NO: 61 - protein, 41 aa, Artificial Sequence
  Epitope
MHVLHLEYNSLVEVNSGSLYGLTALHQLHLSNNSIARIHRK

SEQ ID NO: 62 - protein, 7 aa, Artificial Sequence
  Epitope
GWSFCQK

SEQ ID NO: 63 - protein, 43 aa, Artificial Sequence
  Epitope
GLRSLRVLDLDHNEISGTIEDTSGAFSGLDSLSKLTLFGNKIK

SEQ ID NO: 64 - protein, 5 aa, Artificial Sequence
  Epitope
DCQLK

SEQ ID NO: 65 - protein, 21 aa, Artificial Sequence
  Epitope
DDFLKPQIITQPETTMAMVGK

SEQ ID NO: 66 - protein, 22 aa, Artificial Sequence
  Epitope
DIRFTCSAASSSSSPMTFAWKK

SEQ ID NO: 67 - protein, 9 aa, Artificial Sequence
  Epitope
KDNEVLTNA

SEQ ID NO: 68 - protein, 19 aa, Artificial Sequence
  Epitope
EGRYQCVITNHFGSTYSHK

SEQ ID NO: 69 - protein, 16 aa, Artificial Sequence
  Epitope
ARLTVNVLPSFTKTPH

SEQ ID NO: 70 - protein, 44 aa, Artificial Sequence
  Epitope
ARLTVNVLPSFTKTPHDITIRTTTVARLECAATGHPNPQIAWQK

SEQ ID NO: 71 - protein, 42 aa, Artificial Sequence
  Epitope
LSEIDPAGFEDLPNLQEVYLNNNELTAVPSLGAASSHVVSLF

SEQ ID NO: 72 - protein, 19 aa, Artificial Sequence
  Epitope
LSEIDPAGFEDLPNLQEVY

SEQ ID NO: 73 - DNA, 24 bp, Artificial Sequence
  forward primer
gacggaattcagtgaggagaacct

SEQ ID NO: 74 - DNA, 24 bp, Artificial Sequence
  reverse primer
caactggtagtggcagcttgtagg

SEQ ID NO: 75 - DNA, 24 bp, Artificial Sequence
  forward primer
tcacaaggaacattgtctgaacca

SEQ ID NO: 76 - DNA, 24 bp, Artificial Sequence
  reverse primer
gcctgatctaacacatcctcctca

SEQ ID NO: 77 - DNA, 24 bp, Artificial Sequence
  forward primer
cagcaccttgagctgaacagaaac

SEQ ID NO: 78 - DNA, 24 bp, Artificial Sequence
  reverse primer
ccagcctttggtaatctcggttag

SEQ ID NO: 79 - DNA, 24 bp, Artificial Sequence
  forward primer
ctttcacctatcccacccttatcc

SEQ ID NO: 80 - DNA, 24 bp, Artificial Sequence
  reverse primer
attcatctacggtccacactgctc

SEQ ID NO: 81 - DNA, 20 bp, Artificial Sequence
  forward primer
ggcgtcatggtgggcatggg

SEQ ID NO: 82 - DNA, 20 bp, Artificial Sequence
  reverse primer
atggcgtggggaagggcgta

SEQ ID NO: 83 - protein, 8 aa, Artificial Sequence
  GTC210-01 heavy chain CDR1
GFTFSGYD

SEQ ID NO: 84 - protein, 9 aa, Artificial Sequence
  GTC210-01 heavy chain CDR2
LIYPDSGNK

SEQ ID NO: 85 - protein, 14 aa, Artificial Sequence
  GTC210-01 heavy chain CDR3
ARDAGLSWAGAFDY

SEQ ID NO: 86 - protein, 8 aa, Artificial Sequence
  GTC210-01 light chain CDR1
SSNIGSNY

SEQ ID NO: 87 - protein, 3 aa, Artificial Sequence
  GTC210-01 light chain CDR2
SDS

SEQ ID NO: 88 - protein, 11 aa, Artificial Sequence
  GTC210-01 light chain CDR3
GSWDYSLSAYV

SEQ ID NO: 89 - protein, 8 aa, Artificial Sequence
  GTC210-02 heavy chain CDR1
GFTFSNYY

SEQ ID NO: 90 - protein, 9 aa, Artificial Sequence
  GTC210-02 heavy chain CDR2
GISPGDSST

SEQ ID NO: 91 - protein, 14 aa, Artificial Sequence
  GTC210-02 heavy chain CDR3
AKGLYSNPNEPFDY

SEQ ID NO: 92 - protein, 8 aa, Artificial Sequence
  GTC210-02 light chain CDR1
SSNIGSNY

SEQ ID NO: 93 - protein, 3 aa, Artificial Sequence
  GTC210-02 light chain CDR2
DDS

SEQ ID NO: 94 - protein, 11 aa, Artificial Sequence
  GTC210-02 light chain CDR3
GTWDYSLNGYV

SEQ ID NO: 95 - protein, 8 aa, Artificial Sequence
  GTC210-03 heavy chain CDR1
GFTFSSYD

SEQ ID NO: 96 - protein, 9 aa, Artificial Sequence
  GTC210-03 heavy chain CDR2
GISPDGSNI

SEQ ID NO: 97 - protein, 20 aa, Artificial Sequence
  GTC210-03 heavy chain CDR3
AKVGLRCRYEACSYAYGMDV

SEQ ID NO: 98 - protein, 8 aa, Artificial Sequence
  GTC210-03 light chain CDR1
SSNIGSNY

SEQ ID NO: 99 - protein, 3 aa, Artificial Sequence
  GTC210-03 light chain CDR2
SDS

SEQ ID NO: 100 - protein, 11 aa, Artificial Sequence
  GTC210-03 light chain CDR3
ATWDSSLNGYV

SEQ ID NO: 101 - protein, 8 aa, Artificial Sequence
  GTC210-04 heavy chain CDR1
GFTFSNYD

SEQ ID NO: 102 - protein, 9 aa, Artificial Sequence
  GTC210-04 heavy chain CDR2
SISPSSGSI

SEQ ID NO: 103 - protein, 14 aa, Artificial Sequence
  GTC210-04 heavy chain CDR3
AKDLDAFWRPSFDY

SEQ ID NO: 104 - protein, 8 aa, Artificial Sequence
  GTC210-04 light chain CDR1
SSNIGNNN

SEQ ID NO: 105 - protein, 3 aa, Artificial Sequence
  GTC210-04 light chain CDR2
SDS

SEQ ID NO: 106 - protein, 11 aa, Artificial Sequence
  GTC210-04 light chain CDR3
GSWDDSLSAYV

SEQ ID NO: 107 - protein, 8 aa, Artificial Sequence
  GTC110-01 heavy chain CDR1
GFTFSDYD

SEQ ID NO: 108 - protein, 9 aa, Artificial Sequence
  GTC110-01 heavy chain CDR2
WISHGGGSI

SEQ ID NO: 109 - protein, 20 aa, Artificial Sequence
  GTC110-01 heavy chain CDR3
ARGLGLCKTGLCYYYDAMDV

SEQ ID NO: 110 - protein, 8 aa, Artificial Sequence
  GTC110-01 light chain CDR1
SSNIGNNS

SEQ ID NO: 111 - protein, 3 aa, Artificial Sequence
  GTC110-01 light chain CDR2
ADN

SEQ ID NO: 112 - protein, 11 aa, Artificial Sequence
  GTC110-01 light chain CDR3
AAWDSSLSAYV

SEQ ID NO: 113 - protein, 8 aa, Artificial Sequence
  GTC110-02 heavy chain CDR1
GFTFSDYY

SEQ ID NO: 114 - protein, 9 aa, Artificial Sequence
  GTC110-02 heavy chain CDR2
GISHDSGSK

SEQ ID NO: 115 - protein, 9 aa, Artificial Sequence
  GTC110-02 heavy chain CDR3
ARHWTTFDY

SEQ ID NO: 116 - protein, 8 aa, Artificial Sequence
  GTC110-02 light chain CDR1
SSNIGSNN

SEQ ID NO: 117 - protein, 3 aa, Artificial Sequence
  GTC110-02 light chain CDR2
ANS

SEQ ID NO: 118 - protein, 11 aa, Artificial Sequence
  GTC110-02 light chain CDR3
GAWDYSLSAYV

SEQ ID NO: 119 - protein, 8 aa, Artificial Sequence
  GTC110-03 heavy chain CDR1
GFTFSNYA

SEQ ID NO: 120 - protein, 9 aa, Artificial Sequence
  GTC110-03 heavy chain CDR2
AIYPGGGSI

SEQ ID NO: 121 - protein, 20 aa, Artificial Sequence
  GTC110-03 heavy chain CDR3
ARDILPCPWGRCYYDYAMDV

SEQ ID NO: 122 - protein, 8 aa, Artificial Sequence
  GTC110-03 light chain CDR1
SSNIGSNT

SEQ ID NO: 123 - protein, 3 aa, Artificial Sequence
  GTC110-03 light chain CDR2
ADN

SEQ ID NO: 124 - protein, 11 aa, Artificial Sequence
  GTC110-03 light chain CDR3
GTWDYSLSGYV

SEQ ID NO: 125 - protein, 8 aa, Artificial Sequence
  GTC110-04 heavy chain CDR1
GFTFSNYA

SEQ ID NO: 126 - protein, 9 aa, Artificial Sequence
  GTC110-04 heavy chain CDR2
VISHGGGST

SEQ ID NO: 127 - protein, 20 aa, Artificial Sequence
  GTC110-04 heavy chain CDR3
ARVISNCHLGVCYYSNGMDV

SEQ ID NO: 128 - protein, 8 aa, Artificial Sequence
  GTC110-04 light chain CDR1
SSNIGNND

SEQ ID NO: 129 - protein, 3 aa, Artificial Sequence
  GTC110-04 light chain CDR2
SDS

SEQ ID NO: 130 - protein, 11 aa, Artificial Sequence
  GTC110-04 light chain CDR3
GTWDYSLSGYV

SEQ ID NO: 131 - protein, 121 aa, Artificial Sequence
  GTC210-01 heavy chain_variable region
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSS

SEQ ID NO: 132 - protein, 110 aa, Artificial Sequence
  GTC210-01 light chain_variable region
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVL

SEQ ID NO: 133 - protein, 121 aa, Artificial Sequence
  GTC210-02 heavy chain_variable region
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSS

SEQ ID NO: 134 - protein, 110 aa, Artificial Sequence
  GTC210-02 light chain_variable region
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVL

SEQ ID NO: 135 - protein, 127 aa, Artificial Sequence
  GTC210-03 heavy chain_variable region
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSS

SEQ ID NO: 136 - protein, 110 aa, Artificial Sequence
  GTC210-03 light chain_variable region
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVL

SEQ ID NO: 137 - protein, 121 aa, Artificial Sequence
  GTC210-04 heavy chain_variable region
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSS

SEQ ID NO: 138 - protein, 110 aa, Artificial Sequence
  GTC210-04 light chain_variable region
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVL

SEQ ID NO: 139 - protein, 127 aa, Artificial Sequence
  GTC110-01 heavy chain_variable region
EVQLLESGGGLVQPGGSLRLSCAASGFTFSDYDMSWVRQVPGKGLEWVSWISHGGGSIYYADSVKGRFTISRDNSKNTLYLQMNSLRAEDTAVYYCARGLGLCKTGLCYYYDAMDVWGQGTLVTVSS

SEQ ID NO: 140 - protein, 110 aa, Artificial Sequence
  GTC110-01 light chain_variable region
QSVLTQPPSASGTPGQRVTISCTGSSSNIGNNSVTWYQQLPGTAPKLLIYADNNRPSGVPDRFSGSKSGTSASLAISGLRSEDEADYYCAAWDSSLSAYVFGGGTKLTVL

SEQ ID NO: 141 - protein, 116 aa, Artificial Sequence
  GTC110-02 heavy chain_variable region
EVQLLESGGGLVQPGGSLRLSCAASGFTFSDYYMSWVRQAPGKGLEWVSGISHDSGSKYYADSVKGRFTISRDNSKNTLYLQMNSLRAEDTAVYYCARHWTTFDYWGQGTLVTVSS

SEQ ID NO: 142 - protein, 110 aa, Artificial Sequence
  GTC110-02 light chain_variable region
QSVLTQPPSASGTPGQRVTISCSGSSSNIGSNNVTWYQQLPGTAPKLLIYANSNRPSGVPDRFSGSKSGTSASLAISGLRSEDEADYYCGAWDYSLSAYVFGGGTKLTVL

SEQ ID NO: 143 - protein, 127 aa, Artificial Sequence
  GTC110-03 heavy chain_variable region
EVQLLESGGGLVQPGGSLRLSCAASGFTFSNYAMSWVRQAPGKGLEWVSAIYPGGGSIYYADSVKGRFTISRDNSKNTLYLQMNSLRAEDTAVYYCARDILPCPWGRCYYDYAMDVWGQGTLVTVSS

SEQ ID NO: 144 - protein, 110 aa, Artificial Sequence
  GTC110-03 light chain_variable region
QSVLTQPPSASGTPGQRVTISCSDSSSNIGSNTVSWYQQLPGTAPKLLIYADNNRPSGVPDRFSGSKSGTSASLAISGLRSEDEADYYCGTWDYSLSGYVFGGGTKLTVL

SEQ ID NO: 145 - protein, 127 aa, Artificial Sequence
  GTC110-04 heavy chain_variable region
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSS

SEQ ID NO: 146 - protein, 110 aa, Artificial Sequence
  GTC110-04 light chain_variable region
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVL

SEQ ID NO: 147 - protein, 451 aa, Artificial Sequence
  GTC210-01 heavy chain_mouse IgG2 Fc_full sequence
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 148 - protein, 217 aa, Artificial Sequence
  GTC210-01 light chain_mouse IgG2 Fc_full sequence
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 149 - protein, 451 aa, Artificial Sequence
  GTC210-02 heavy chain_mouse IgG2 Fc_full sequence
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 150 - protein, 217 aa, Artificial Sequence
  GTC210-02 light chain_mouse IgG2 Fc_full sequence
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 151 - protein, 457 aa, Artificial Sequence
  GTC210-03 heavy chain_mouse IgG2 Fc_full sequence
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 152 - protein, 217 aa, Artificial Sequence
  GTC210-03 light chain_mouse IgG2 Fc_full sequence
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 153 - protein, 449 aa, Artificial Sequence
  GTC210-04 heavy chain_mouse IgG2 Fc_full sequence
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 154 - protein, 217 aa, Artificial Sequence
  GTC210-04 light chain_mouse IgG2 Fc_full sequence
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 155 - protein, 451 aa, Artificial Sequence
  GTC110-01 heavy chain_mouse IgG2 Fc_full sequence
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 156 - protein, 217 aa, Artificial Sequence
  GTC110-01 light chain_mouse IgG2 Fc_full sequence
QSVLTQPPSASGTPGQRVTISCTGSSSNIGNNSVTWYQQLPGTAPKLLIYADNNRPSGVPDRFSGSKSGTSASLAISGLRSEDEADYYCAAWDSSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 157 - protein, 446 aa, Artificial Sequence
  GTC110-02 heavy chain_mouse IgG2 Fc_full sequence
EVQLLESGGGLVQPGGSLRLSCAASGFTFSDYYMSWVRQAPGKGLEWVSGISHDSGSKYYADSVKGRFTISRDNSKNTLYLQMNSLRAEDTAVYYCARHWTTFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 158 - protein, 217 aa, Artificial Sequence
  GTC110-02 light chain_mouse IgG2 Fc_full sequence
QSVLTQPPSASGTPGQRVTISCSGSSSNIGSNNVTWYQQLPGTAPKLLIYANSNRPSGVPDRFSGSKSGTSASLAISGLRSEDEADYYCGAWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 159 - protein, 457 aa, Artificial Sequence
  GTC110-03 heavy chain_mouse IgG2 Fc_full sequence
EVQLLESGGGLVQPGGSLRLSCAASGFTFSNYAMSWVRQAPGKGLEWVSAIYPGGGSIYYADSVKGRFTISRDNSKNTLYLQMNSLRAEDTAVYYCARDILPCPWGRCYYDYAMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 160 - protein, 217 aa, Artificial Sequence
  GTC110-03 light chain_mouse IgG2 Fc_full sequence
QSVLTQPPSASGTPGQRVTISCSDSSSNIGSNTVSWYQQLPGTAPKLLIYADNNRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 161 - protein, 457 aa, Artificial Sequence
  GTC110-04 heavy chain_mouse IgG2 Fc_full sequence
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 162 - protein, 217 aa, Artificial Sequence
  GTC110-04 light chain_mouse IgG2 Fc_full sequence
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 163 - protein, 458 aa, Artificial Sequence
  GTC110-04 heavy chain_human_full sequence
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKY

SEQ ID NO: 164 - protein, 217 aa, Artificial Sequence
  GTC110-04 light chain_human_full sequence
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC